(19) United States
(12) Patent Application Publication
(10) Pub. No.: US 2021/0355538 A1
(43) Pub. Date: Nov. 18, 2021

(54) USE OF MUCOSAL TRANSCRIPTOMES FOR ASSESSING SEVERITY OF ULCERATIVE COLITIS AND RESPONSIVENESS TO TREATMENT

(71) Applicant: CHILDREN'S HOSPITAL MEDICAL CENTER, Cincinnati, OH (US)

(72) Inventor: Lee Denson, Cincinnati, OH (US)

(21) Appl. No.: 17/286,784

(22) PCT Filed: Oct. 18, 2019
(86) PCT No.: PCT/US2019/057049

§ 371 Date: Apr. 19, 2021

Related U.S. Application Data
(60) Provisional application No. 62/747,792, filed on Oct. 19, 2018.

(51) Int. Cl. C12Q 1/6883; C12Q 1/686

(52) U.S. Cl. CPC C12Q 1/6883; C12Q 2600/158; C12Q 2600/106; C12Q 1/686

(57) ABSTRACT

The present disclosure provides methods for assessing responsiveness or non-responsiveness to a therapeutic agent (e.g., steroid therapy, anti-TNF therapy or anti-integrin α4β7 therapy) in ulcerative colitis (UC) subjects based on gene signatures. The methods may further comprise identifying suitable treatment for the patient based on the gene signatures.

### CROSS-REFERENCE TO RELATED APPLICATIONS

This application claims the benefit of the filing date of U.S. Provisional Application No. 62/747,792, filed Oct. 19, 2018, the entire contents of which are incorporated by reference herein.

### BACKGROUND OF THE INVENTION

Ulcerative colitis (UC) is an episodic inflammatory bowel disease of the colon. The exact etiology of ulcerative colitis (UC) is unknown, but certain factors have been found to be associated with the disease, including genetic factors, immune system reactions, environmental factors, nonsteroidal anti-inflammatory drug (NSAID) use, low levels of antioxidants, psychological stress factors, a smoking history, microbial infection and consumption of milk products. Gene expression is thought to contribute to the overall course of the disease, but also reflects the processes that underlie the clinical expression of active disease and disease in remission. Genetically susceptible individuals have abnormalities of the humoral and cell-mediated immunity and/or generalized enhanced reactivity against commensal intestinal bacteria, and that this dysregulated mucosal immune response predisposes to colonic inflammation.

The treatment of UC is made on the basis of the disease stage (active, remission), extent (proctitis, distal colitis, left-sided colitis, pancolitis), and severity (mild, moderate, severe). In general, it relies on initial medical management with corticosteroids and anti-inflammatory agents, such as sulfasalazine, in conjunction with symptomatic treatment with antidiarrheal agents and rehydration. However, not all patients respond to these regimens. Surgery is contemplated when medical treatment fails or when a surgical emergency (e.g., perforation of the colon) occurs. Surgical options include total colectomy (panproctocolectomy) and ileostomy, total colectomy, and ileoanal pouch reconstruction or ileorectal anastomosis. The loss of clinical response is a challenge that results in further morbidity, reduced quality of life, and increased costs. To date, there is no validated approach for monitoring patient health status while under treatment. Considering the variability in patient response and the frequent occurrence of flares or relapse in disease, finding and validating novel approaches for patient monitoring and self-monitoring holds great promise for improving care as well as patient quality of life.

It is therefore of great interest to develop new approaches for monitoring UC disease severity and predicting responsiveness to treatment.

### SUMMARY OF THE INVENTION

The present disclosure is based on the unexpected discovery of gene signatures, e.g., ulcerative colitis disease occurrence and/or severity signature and corticosteroid responsiveness gene signatures as disclosed herein, which correlate with disease occurrence, severity, and/or patient responsiveness to anti-UC treatment, such as steroid treatment, anti-TNFα treatment, and/or anti-α₄β₇ integrin treatment. Such gene signatures can help determine suitable treatment for UC patients, for example, pediatric UC patients.

Accordingly, one aspect of the present disclosure provides a method for assessing responsiveness to UC therapy (e.g., a steroid therapy such as a corticosteroid therapy, an anti-TNFα therapy, and/or an anti-α₄β₇ integrin therapy) in a subject having ulcerative colitis. The method may comprise: (i) measuring expression levels of a group of genes in a biological sample of a subject having ulcerative colitis, wherein the group of genes consists of two or more genes selected from the genes listed in Table 1; (ii) determining a steroid responsiveness gene signature based on the expression levels of the two or more genes in step (i); and (iii) assessing the subject's responsiveness to a UC therapy based on at least the steroid responsiveness gene signature. In some embodiments, the UC therapy can be a steroid therapy, an anti-TNFα therapy, and/or an anti-α₄β₇ integrin therapy. In particular examples, the UC therapy is a steroid therapy, for example, a corticosteroid therapy.

In some embodiments, the group of genes may comprise at least two genes involved in two different biological pathways, and wherein the two different biological pathways are selected from the group consisting of cytokine activity, CXCR1 interaction, RAGE receptor binding, neutrophil degranulation, granulocyte migration, and response to bacterium. In some examples, the group of genes may comprise at least one gene involved in cytokine activity, one gene involved in CXCR1 interaction, one gene involved in RAGE receptor binding, one gene involved in neutrophil degranulation, one gene involved in granulocyte migration, and one gene involved in response to bacterium. In one particular example, the group of genes comprises DEFB4A, CSF2, CXCR1, S100A9, FCGR3B, OSM, and TREM1. In another particular example, the group of genes consists of all genes listed in Table 1.

The steroid responsiveness gene signature may be determined by a computational analysis. In any of the methods disclosed herein, the steroid responsiveness gene signature can be represented by a score calculated by the computational analysis based on the expression levels of the group of genes. Deviation of the score from a predetermined value indicates the subject's responsiveness or non-responsiveness to the UC therapy (i.e., likely to respond to the UC treatment or unlikely to respond to the treatment). In some embodiments, the subject's responsiveness to the UC therapy comprises Week 4 clinical remission.

In some embodiments, assessment of the subject's responsiveness to the UC therapy (e.g., a steroid therapy such as a corticosteroid threapy) in step (iii) is further based on one or more clinical factors. In some examples, the one or more clinical factors comprise gender, level of rectal eosinophils, and disease severity. In one example, the level of rectal eosinophils is represented by the expression level of ALOX15 in a rectal biopsy sample of the subject.

In some embodiments, any of the methods disclosed herein may further comprise, prior to step (iii), analyzing microbial populations in the biological sample. In some examples, assessment of UC therapy (e.g., steroid therapy such as corticosteroid therapy) responsiveness of the subject in step (iii) can be further based on abundance of disease-associated and beneficial microbial populations in the biological sample.

Any of the methods disclosed herein may further comprise subjecting the subject to a suitable treatment of ulcerative colitis based on the assessment of the subject's responsiveness to the UC therapy determined in step (iii). For example, when the subject is determined to be responsive to the UC treatment, the method may further comprise administering to the subject a steroid, an anti-TNFα agent, an anti-α₄β₇ integrin agent, or a combination thereof, for treating ulcerative colitis. In some examples, a steroid such as a corticosteroid is given to the subject. Alternatively, when the subject is determined to be non-responsive to the treatment, the method may further comprise administering to the subject a non-steroid therapeutic agent for treating ulcerative colitis. In some examples, the non-steroid therapeutic agent is not an anti-anti-TNFα agent and/or not an anti-α₄β₇ integrin agent.

In another aspect, provided herein is a method for identifying a subject having or at risk for ulcerative colitis (UC), the method comprising: (i) measuring expression levels of (a) one or more genes involved in mitochondrial function, (b) one or more genes involved in the Kreb cycle, or (c) a combination of (a) and (b) in a biological sample of a subject; (ii) determining a UC disease occurrence and/or severity gene signature based on the expression levels of the genes in step (i); and (iii) assessing UC occurrence and/or severity of the subject based on the gene signature determined in step (ii).

In some embodiments, the one or more genes involved in mitochondrial function comprises PPARGC1A (PGC-1α), MT-CO1, COX5A, a Complex I gene, a Complex III gene, a Complex IV gene, a Complex V gene, or a combination thereof. In some examples, step (i) involves measuring the expression level of PPARGC1A (PGC-1α) in the biological sample. Alternatively or in addition, step (i) involves measuring the levels of MT-CO1+ and/or COX5A+ cells in the biological sample. Further, step (i) may involve measuring the level of the Complex I gene, the Complex III gene, the Complex IV gene, the Complex V gene, or a combination thereof. Exemplary Complex I genes include MT-ND1, MT-ND2, MT-ND3, MT-ND4, MT-ND4L, MT-ND5, and/or MT-ND6. Exemplary Complex III gene can be MT-CYB. Exemplary Complex IV genes include MT-COL MT-CO2, and/or MT-CO3. Exemplary Complex V genes include MT-ATP6 and/or MT-ATPS. See also FIG. 2A.

The UC disease occurrence and/or severity gene signature can be determined by a computational analysis. In some embodiments, when the subject is identified as having or at risk for UC, the method may further comprise subjecting the subject to a treatment of UC. In some embodiments, the subject is a UC patient and is identified as having an active disease, the method may further comprise subjecting the subject to a treatment of UC (e.g., a treatment different from a current treatment performed on the subject).

In some embodiments, the subject analyzed in any of the methods disclosed herein can be a human pediatric patient having ulcerative colitis. In some examples, the subject may be free of a prior UC treatment, for example, a prior steroid treatment.

In any of the methods disclosed herein, the biological sample can be a rectal biopsy sample of the subject. In some examples, the expression levels of the genes can be measured by RT-PCR and microarray analysis.

Also within the scope of the present disclosure are suitable anti-UC therapeutic agents (e.g., a steroid agent such as a corticosteroid agent or a non-steroid agent) for use in treating a UC patient who is identified as responsive or not responsive to a steroid therapy, an anti-TNFa treatment, and/or an anti-α4β7 integrin treatment based on the corticosteroid responsiveness gene signature disclosed herein, or uses of the anti-UC therapeutic agents for manufacturing a medicament for the intended medical use. In addition, provided herein are suitable anti-UC therapeutic agents as disclosed herein for use in treating a subject who is identified as having the disease, at risk for the disease, or in an active disease stage based on the disease occurrence and/or severity gene signature as disclosed herein, or uses of such suitable anti-UC therapeutic agents for manufacturing a medicament for the intended therapy.

The details of one or more embodiments of the invention are set forth in the description below. Other features or advantages of the present invention will be apparent from the following drawings and detailed description of several examples, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is chart showing a computational deconvolution of cell subset proportions in 206 UC patients and 20 healthy controls.

FIGS. 2A-2M include diagrams showing colonic mitochondrionpathy with a robust gene signature for reduced rectal mitochondrial energy functions in US. FIG. 2A: a bar graph showing that 13 mitochondrial encoded genes are down-regulated in UC vs. control with their fold change, FDR corrected p-value, and associated mitochondrial complex as indicated. FIG. 2B: a graph showing High-Resolution Respirometry performed on fresh colon biopsies (5 control, 9 with active UC, and 9 with inactive UC) using the Oroboros O2k modular system to evaluate the activity of Complex I. FIG. 2C: a graph showing High-Resolution Respirometry performed on fresh colon biopsies (5 control, 9 with active UC, and 9 with inactive UC) using the Oroboros O2k modular system to evaluate the activity of Complex II of the electron transport chain. FIG. 2D: a graph showing JC1 staining and FACS analysis to define the mitochondrial membrane potential of EpCAM⁺ epithelial cells. FIG. 2E: a graph showing JC1 staining and FACS analysis to define the mitochondrial membrane potential of CD45⁺ leukocytes isolated from colon biopsies (7 controls, 6 active UC, and 7 with inactive UC, 85-99% viability). FIG. 2F: a box plot showing colon PPARGC1A (PGC-1α) expression for the PROTECT cohort in normalized values was plotted after stratifying the samples as indicated. FIG. 2G: a box plot showing the Krebs cycle TCA gene signature PCA PC1 for the PROTECT cohort. FIG. 2H: a box plot showing colon PPARGC1A (PGC-1α) expression for the RISK cohort in [Transcripts per Million (TPM) values] in normalized values was plotted after stratifying the samples as indicated. FIG. 2I: a box plot showing the Krebs cycle TCA gene signature PCA PC1 for the RISK cohort. FIG. 2J: a box plot showing colon PPARGC1A (PGC-1α) expression for the adult UC cohort (GSE5907112) in normalized values was plotted after stratifying the samples as indicated. FIG. 2K: a box plot showin the Krebs cycle TCA gene signature PCA PC1 for the GSE59071 cohort. FIG. 2L: a photo showing immunohistochemical staining of representative rectal MT-CO1 and COX5A immunohistochemistry (complex IV) for Ctl (n=14) inactive (n=10) and active UC (n=11) with moderate Mayo endoscopic subscore and moderate PUCAI. Scale bar represents 50 micron. FIG. 2M: two graphs showing the frequency of MT-CO1 positive (top panel) and COX5A positive (bottom panel) epithelial cells out of the total epithelial cells for controls, inactive UC, and active UC. Box and whisker plot with central line indicating median, box ends representing upper and lower quartile, and whisker represent 10-90 percentile. Kruskal-Wallis with Dunn's Multiple Comparison or ANOVA with false discovery rate (FDR) was used *All 2-sided P<0.05, **P<0.01, ***P<0.001, ****P<0.0001. UC: ulcerative colitis; L2 cCD: colon-only Crohn's disease; L3 iCD: ileo-colonic Crohn's disease.

FIGS. 3A-3D include diagrams showing that disease severity is linked to adenoma/adenocarcinoma and innate immune pathways. FIG. 3A: a chart showing a computational deconvolution of cell subset proportions in controls and UC patients stratified by endoscopic severity mayo subscore. Differences [ANOVA with with FDR<0.05 (*)] between mayo 3 (severe, n=71) and 1 (mild, n=27) are shown. FIG. 3B: two graphs showing immune cell type enrichment of up-regulated genes for (Top) 5296 core UC and (Bottom) 712 UC severity genes using the Immunological Genome Project data series as a reference through ToppGene. Enrichment for a given immune cell class is illustrated by colored bars on the x axis, with the significance for each individual cell subtype within the class shown as the −log 10(P value) on the y axis. DC; Dendritic cells. FIG. 3C: a graph showing the frequency (percent of patient of the total per group) of Mild (n=54) and moderate-severe (n=152) patients across histology severity scores. FIG. 3D: a graph showing the distribution of moderate-severe patients who did or did not achieve week 4 (WK4) remission across histology severity scores. UC: ulcerative colitis.

FIGS. 4A-4I include diagrams showing a rectal gene signature is associated with response to UC induction therapy and microbial shift. FIG. 4A: a box plot showing samples loading PC1 (Z score) values of the corticosteroid responsiveness gene signature are shown for controls and the discovery cohort of 152 moderate-severe UC patients stratified by WK4 clinical remission (R). FIG. 4B: a box plot showing samples loading PC1 (Z score) values of the corticosteroid responsiveness gene signature are shown for controls and the discovery cohort of 152 moderate-severe UC patients stratified by mucosal healing (fecal calprotectin <250 mcg/gm). FIG. 4C: a box plot showing samples loading PC1 values derived from an independent 3′UTR Lexogen mRNASeq platform for the discovery cohort and an independent validation cohort stratified by WK4 clinical remission (R). FIG. 4D: a box plot showing samples loading PC1 values derived from an independent 3′UTR Lexogen mRNASeq platform for the discovery cohort and an independent validation cohort stratified by) mucosal healing for the validation cohort. FIG. 4E: a box plot showing samples loading PC1 values including controls and the GSE1687920 data set of UC treated with anti-TNF. FIG. 4F: a box plot showing and samples loading PC1 values including controls and the GSE7366123 dataset of UC treated with anti-integrin α4β7. R: mucosal healing defined by colonoscopy. FIG. 4G: a diagram showing the functional annotation enrichment analyses of the corticosteroid responsiveness gene signature and the top 50 genes that were differentially expressed in pre-treatment colon biopsies of anti-TNF refractory vs responsive UC patients. Genes are denoted in hexagons and biologic functions denoted in squares; connections to each signature are as shown. FIG. 4H: a heat map summarizing Spearman similarity measures between microbial abundances and gene expression using hierarchical all-against-all association. *False discovery rate <0.2. Blue and red indicates negative and positive associations respectively. FIG. 4I: a graphical summary of the cohort and main findings showing determining the corticosteroid responsiveness gene signature PC1 is a significant predictor of corticosteroid responsiveness than clinical factors alone.

### DETAILED DESCRIPTION OF THE INVENTION

Ulcerative colitis (UC) is a chronic relapsing-remitting inflammatory bowel disease (IBD) diagnosed primarily in young individuals. The disease burden has increased with globalization; newly industrialized countries show the greatest increase in incidence and the highest prevalence is recorded in Western countries. Kaplan et al., Gastroenterology 152: 313-321 (2017); and Peery et al., Gastroenterology 143: 1179-1187 (2012). Disease severity and treatment response are strikingly heterogeneous with some patients quickly and continually responding to initial therapies while others experience ongoing inflammation ultimately requiring surgical resection of the affected bowel. Hyams et al., Lancet Gastroenterol Hepatol, doi:10.1016/52468-1253(17)30252-2 (2017); and Hyams et al., The Journal of pediatrics 129: 81-88 (1996). Greater understanding of individualized pathways driving clinical and mucosal severity and response to therapy, and the clinical translation of these data, is needed to proactively identify targeted therapeutic approaches.

To improve the understanding of UC pathogenesis and its potential clinical personalized translation, a standardized approach was applied to a large, multicenter inception cohort that collected samples before treatment initiation, and included subjects representing the full spectrum of disease severities. The Predicting Response to Standardized Pediatric Colitis Therapy (PROTECT) study included 428 UC patients from 29 pediatric gastroenterology centers in North America. Hyams et al., 2017. At diagnosis, disease was clinically and endoscopically graded, rectal biopsy histology was centrally read, and clinical and demographic data were recorded. Patients were assigned a specific standardized initial therapy with mesalamine or corticosteroids, and outcomes were recorded. Boyle et al., Am J Surg Pathol 41:1491-1498 (2017). Rectal biopsies from a representative sub-cohort of 206 patients underwent high throughput RNA sequencing (RNAseq) prior to medical therapy, representing the largest UC transcriptomic cohort to date. Robust gene expression and pathways that are linked to UC pathogenesis, severity, response to corticosteroid therapy, and gut microbiota, which provide new insights into molecular mechanisms driving disease course.

Based on the gene expression analysis disclosed herein, gene signatures correlating to UC patients' responsiveness/non-responsiveness to certain UC treatment, or gene signatures correlating to UC disease occurrence and/or severity have been identified and reported herein. Such gene signatures can be relied on to determine suitable treatment or adjust current UC therapy for subjects who need the treatment.

### I. Assessing Therapeutic Responsiveness/Non-Responsiveness in Ulcerative Colitis Patients

One aspect of the present disclosure relates to methods for assessing responsiveness or non-responsiveness of a US patient (e.g., a human UC patient such as a human pediatric UC patient) would be responsive or non-responsive to a therapeutic agent (e.g., steroid therapy such as a corticosteroid therapy, anti-TNF therapy, and/or anti-α4β7 integrin therapy) based on a corticosteroid responsiveness gene signature as disclosed herein. As used herein, assessing “responsiveness” or “non-responsiveness” to a therapeutic agent refers to the determination of the likelihood of a subject for responding or not responding to the therapeutic agent.

A. Steroid/Corticosteroid Responsiveness Gene Signatures

A gene signature refers to a characteristic expression profile of a single or a group of genes that is indicative of an altered or unaltered biological process, medical condition, or a patient's responsiveness/non-responsiveness to a specific therapy. The steroid/corticosteroid responsiveness gene signatures disclosed herein encompass characteristic expression profiles of two or more genes listed in Table 1 below, which are identified as differentially expressed in baseline rectal biopsies between moderate-severe UC patients who did or did not achieve clinical remission at week 4 (WK4 outcome), irrespective of initial corticosteroid status. See Example below.

TABLE 1
Corticosteroid Responsiveness Genes

| | p (Corr) | FC | | |
|---|---|---|---|---|
| | [Responders] vs | [Responders] vs | [Responders] vs | Involved Biological |
| Gene | [non-Responders] | [non-Responders] | [non-Responders] | Pathways |
| SPRR2A | 0.00156928 | −3.9108756 | down | Peptide cross-linking |
| SPRR1B | 0.002087139 | −3.7260742 | down | Peptide cross-linking |
| DEFB4A | 7.68E−04 | −2.984436 | down | Response to |
| | | | | bacterium |
| REG1A | 0.004578535 | −2.609347 | down | Response to |
| | | | | bacterium |
| SPRR3 | 0.009392924 | −2.6067 | down | Peptide cross-linking |
| S100A12 | 0.002074444 | −2.5414026 | down | RAGE receptor |
| | | | | binding |
| MCEMP1 | 0.002074444 | −2.1394966 | down | Neutrophil |
| | | | | degranulation |
| CSF3 | 0.003326554 | −2.1094072 | down | Cytokine activity/ |
| | | | | granulocyte |
| | | | | migration |
| KRT6A | 0.006396802 | −2.0945237 | down | Defense response |
| S100A8 | 0.002074444 | −2.0935678 | down | RAGE receptor |
| | | | | binding |
| PROK2 | 0.002653977 | −2.0545259 | down | Defense response |
| BEAN1 | 3.16E−04 | −2.0187218 | down | NA |
| FCAR | 0.001580823 | −1.9877136 | down | Granulocyte |
| | | | | activation |
| SAA4 | 0.003016525 | −1.9668278 | down | Defense response |
| CSF2 | 0.002074444 | −1.9444672 | down | CXCR1 interaction |
| | | | | Cytokine activity |
| HCAR3 | 0.004065251 | −1.9289553 | down | Signaling receptor |
| | | | | activity |
| TCN1 | 0.002653977 | −1.8557938 | down | Granulocyte |
| | | | | activation |
| SELE | 0.00319337 | −1.8517934 | down | Response to |
| | | | | bacterium |
| AQP9 | 0.002944914 | −1.8379968 | down | Response to |
| | | | | bacterium |
| KRT6B | 0.013021237 | −1.8308139 | down | Epithelial cell |
| | | | | differentiation |
| CXCR1 | 0.002428178 | −1.819651 | down | CXCR1 interaction |
| SFRP2 | 0.009444999 | −1.8115587 | down | Cytokine activity |
| S100A9 | 0.002365904 | −1.8092808 | down | RAGE receptor |
| | | | | binding |
| FPR2 | 0.00246693 | −1.7929862 | down | RAGE receptor |
| | | | | binding |
| TNIP3 | 0.003344591 | −1.7910203 | down | Neutrophil |
| | | | | degranulation |
| LYPD1 | 7.68E−04 | −1.789777 | down | Defense response |
| GLT1D1 | 0.001718353 | −1.7798088 | down | Human mesenchymal |
| | | | | stem cells |
| INHBA | 0.00156928 | −1.7783887 | down | Cytokine activity |
| MMP10 | 0.002365904 | −1.7751089 | down | Endopeptidase |
| | | | | activity |
| FAM83A | 0.003034759 | −1.7719635 | down | NA |
| FCGR3B | 0.003402458 | −1.7679293 | down | Response to |
| | | | | bacterium |
| IL6 | 0.005562924 | −1.7658511 | down | Cytokine activity |
| CMTM2 | 0.004508805 | −1.7525514 | down | CXCR1 interactions |
| APOBEC3A | 0.002365904 | −1.7513928 | down | Defense response |
| SAA2 | 0.002980155 | −1.7481767 | down | Defense response |
| CLEC4D | 0.004356155 | −1.7351102 | down | Response to |
| | | | | bacterium/ |
| | | | | neutrophil |
| | | | | degranulation |
| PPBP | 0.002944914 | −1.7346658 | down | CXCR1 interactions/ |
| | | | | neutrophil |
| | | | | degranulation |
| OSM | 0.005978393 | −1.7221636 | down | Cytokine activity |
| IL1A | 0.00156928 | −1.7206603 | down | Cytokine activity |
| SAA1 | 0.006561303 | −1.6982508 | down | Granulocyte |
| | | | | migration |
| ADAMTS4 | 0.003034759 | −1.6941336 | down | Defense response |
| KCNJ15 | 0.003698882 | −1.6817317 | down | Ion transport |
| IFNG | 0.002653977 | −1.6626679 | down | Response to |
| | | | | bacterium/cytokine |
| | | | | activity |
| SLC6A14 | 0.00237334 | −1.6606127 | down | Ion transport |
| ENKUR | 0.001572466 | −1.6549691 | down | Secretory granule |
| ANGPTL4 | 0.003035548 | −1.6482337 | down | Regulation of |
| | | | | angiogenesis |
| CLDN14 | 0.002365904 | −1.6469289 | down | Cell adhesion |
| MMP1 | 0.009392924 | −1.6407094 | down | Endopeptidase |
| | | | | activity |
| HCAR2 | 0.01060739 | −1.6310117 | down | Signaling receptor |
| | | | | activity |
| CXCL6 | 0.002428178 | −1.6283742 | down | Cytokine activity/ |
| | | | | CXCr1 interactions |
| GPR84 | 0.002944914 | −1.627954 | down | Granulocyte |
| | | | | migration |
| ADGRF1 | 0.001099469 | −1.62272 | down | Cyclase activity |
| CLDN1 | 0.001537864 | −1.6222031 | down | Cell adhesion |
| TREM1 | 0.004578535 | −1.622006 | down | Response to |
| | | | | bacterium |
| SLC11A1 | 0.004065251 | −1.621678 | down | Granulocyte |
| | | | | migration |
| CXCL17 | 0.013513103 | −1.6202309 | down | Cytokine activity |
| CD274 | 7.68E−04 | −1.6180012 | down | T cell proliferation |
| CXCR2 | 0.004679616 | −1.6176988 | down | Cytokine activity |
| | | | | CXCR1 interaction |
| CXCL8 | 0.007517018 | −1.6047142 | down | Cytokine activity/ |
| | | | | CXCR1 interactions |
| NFE2 | 0.004575382 | −1.596516 | down | Wound healing |
| IL1B | 0.005381064 | −1.5936643 | down | Cytokine activity |
| CD300E | 0.005559608 | −1.5934315 | down | Defense response |
| AGT | 0.002087139 | −1.5882807 | down | Defense response |
| SAA2- | 0.013480227 | −1.5872025 | down | Defense response |
| SAA4 | | | | |
| ITGA2 | 0.001999571 | −1.5804726 | down | Defense response |
| HP | 0.012289889 | −1.5748503 | down | Response to |
| | | | | bacterium |
| FPR1 | 0.003521594 | −1.5738393 | down | RAGE receptor |
| | | | | binding |
| CSF3R | 0.003227453 | −1.5660037 | down | Granulocyte |
| | | | | migration |
| C2CD4A | 0.002924505 | −1.5574645 | down | Defense response |
| VSIG1 | 0.013231894 | −1.556089 | down | Epithelial cell |
| | | | | differentiation |
| WISP1 | 0.002428178 | −1.5530255 | down | NA |
| MMP3 | 0.018594624 | −1.5508299 | down | Endopeptidase |
| | | | | activity |
| STC1 | 0.008923925 | −1.5496097 | down | Cell migration |
| CXCL11 | 0.020787785 | −1.5493516 | down | Cytokine activity/ |
| | | | | CXCR1 interactions |
| LILRA6 | 0.00326201 | −1.5465705 | down | NA |
| CXCL10 | 0.006396802 | −1.5463748 | down | Cytokine activity |
| IL11 | 0.03413381 | −1.544713 | down | Cytokine activity/ |
| | | | | neutrophil |
| | | | | degranulation |
| GAL | 0.004578535 | −1.5393486 | down | Defense response |
| FCN3 | 0.002074444 | −1.5383366 | down | Defense response |
| FOSL1 | 0.007078409 | −1.5379435 | down | Defense response |
| C4BPA | 0.015581701 | −1.536158 | down | Defense response |
| RND1 | 7.68E−04 | −1.5356064 | down | Cell migration |
| CLEC5A | 0.00517247 | −1.5248593 | down | Neutrophil |
| | | | | degranulation |
| PLAU | 0.00156928 | −1.5231256 | down | Response to |
| | | | | bacterium/ |
| | | | | granulocyte |
| | | | | migration |
| PLLP | 7.68E−04 | 1.5045084 | up | Ion transport |
| FRMD1 | 0.013110096 | 1.5066905 | up | NA |
| UGT1A8 | 0.022624416 | 1.513314 | up | Lipid metabolic |
| | | | | process |
| GLDN | 0.025545727 | 1.5393035 | up | Cell adhesion |
| FCER1A | 7.68E−04 | 1.543177 | up | Immunoglobulin |
| | | | | binding |
| SLC26A2 | 0.010915723 | 1.552315 | up | Ion transport |
| CA2 | 0.003536249 | 1.5626011 | up | Secretion |
| FABP1 | 0.001921544 | 1.6130058 | up | Fatty acid binding |
| TMEM72 | 0.04337912 | 1.6157596 | up | NA |
| ABCG2 | 0.004270176 | 1.6181817 | up | Cation homeostasis |
| RBP2 | 0.019890927 | 1.6233547 | up | Lipid metabolic |
| | | | | process |
| IGSF9 | 0.001099469 | 1.6254493 | up | Cell adhesion |
| TRPM6 | 0.006396802 | 1.630646 | up | Ion transport |
| SLC30A10 | 0.003674042 | 1.6400309 | up | Ion transport |
| GLRA2 | 0.016535196 | 1.6499856 | up | Ion transport |
| HMGCS2 | 0.02028233 | 1.6754341 | up | Lipid metabolic |
| | | | | process |
| USP2 | 7.68E−04 | 1.7025073 | up | Endopeptidase |
| | | | | activity |
| CKB | 0.002168184 | 1.709176 | up | Anion homeostasis |
| CD177 | 0.031690687 | 1.7167165 | up | Defense response |
| SLC26A3 | 0.002087139 | 1.8151755 | up | Cation homeostasis |
| SULT1A2 | 0.00156928 | 1.8156435 | up | Response to lipid |
| CHP2 | 0.00551686 | 1.841157 | up | Cation homeostasis |
| PLA2G12B | 0.013021237 | 1.8696988 | up | Ion transport |
| VSTM2A | 0.008197488 | 1.8899074 | up | Regulation of cell |
| | | | | proliferation |
| TMIGD1 | 0.009042374 | 1.9924744 | up | Cell migration |
| GUCA2A | 0.003801226 | 2.0073035 | up | Cyclase activity |
| PCK1 | 0.003324416 | 2.2008889 | up | Leukocyte migration |
| GUCA2B | 0.002365904 | 2.3606446 | up | Cyclase activity |
| CA1 | 0.003227453 | 2.760886 | up | Ion transport |
| OTOP2 | 0.001999571 | 2.7846637 | up | Ion transport |
| AQP8 | 0.00156928 | 5.435324 | up | Secretion |

Table 1 above lists genes that are differentially expressed (up or down as indicated) in responders versus non-responders, as well as the potential biological pathways those genes involve, including cytokine activity, defense response, response to bacterium, ion transport and homeostasis, CXCR1 interaction, RAGE receptor binding, neutrophil degranulation, granulocyte migration and activation, endopeptidase activity, peptide cross-linking, cell adhesion, cyclase activity, lipid metabolic process, signaling receptor activity, and epithelial cell differentiation.

The corticosteroid responsiveness gene signature may represent the expression profile of at least two genes selected from Table 1, for example, at least 3 genes, 4, genes, 5 genes, 6 genes, 7 genes, 8 genes, 9 genes, 10 genes, 15 genes, 20 genes, 25 genes, or more. In some examples, the corticosteroid responsiveness gene signature may comprise multiple up-regulated genes as indicated in Table 1. In other examples, the corticosteroid responsiveness gene signature may comprise multiple down-regulated genes as indicated in Table 1. In yet other examples, the corticosteroid responsiveness gene signature may comprise both up-regulated and down-regulated genes as indicated in Table 1. In specific examples, the corticosteroid responsiveness gene signature comprises all genes listed in Table 1.

In some embodiments, the corticosteroid responsiveness gene signature may comprise multiple genes involved in multiple biological pathways, for example, 2 biological pathways, 3 biological pathways, 4 biological pathways, 5 biological pathways, 6 biological pathways, 7 biological pathways, 8 biological pathways, 9 biological pathways, 10 biological pathways, 11 biological pathways, 12 biological pathways, 13 biological pathways, 14 biological pathways, or 15 biological pathways.

In some examples, the corticosteroid responsiveness gene signature comprises at least one gene that is involved in cytokine activity. Non-limiting examples of genes involved in cytokine activity to be used as biomarkers in the methods described herein include CSF3 (e.g., GenBank Accession Nos. NP_000750.1 and NM_000759.3), CSF2 (e.g., GenBank Accession Nos. NP_000749.2 and NM_000758.3), SFRP2 (e.g., GenBank Accession Nos. NP_003004.1 and NM_003013.2), INHBA (e.g., GenBank Accession Nos. NP_002183.1 and NM_002192.3), IL6 (e.g., GenBank Accession Nos. NP_000591.1 and NM_000600.4), OSM (e.g., GenBank Accession Nos. NP_001306037.1, NM_001319108.1, NP_065391.1, and NM_020530.5), ILIA (e.g., GenBank Accession NP_000566.3 and NM_000575.4), IFNG (e.g., GenBank Accession Nos. NP_000610.2 and NM_000619.2), CXCL6 (e.g., GenBank Accession Nos. NP_002984.1 and NM_002993.3), CXCL17 (e.g., GenBank Accession Nos. NP_940879.1 and NM_198477.2), CXCR2 (e.g., GenBank Accession Nos. NP_001161770.1 and NM_001168298.1), CXCL8 (e.g., GenBank Accession Nos. NP_000575.1 and NM_000584.3), IL1B (e.g., GenBank Accession Nos. NP_000567.1 and NM_000576.2), CXCL11 (e.g., GenBank Accession Nos. NP_001289052.1 and NM_001302123.1), CXCL10 (e.g., GenBank Accession Nos. NP_001556.2 and NM_001565.3), and IL11 (e.g., GenBank Accession No. NP_000632.1 and NM_000641.3). In specific examples, the gene(s) involved in cytokine activity is CSF2, OSM, or a combination thereof.

In some examples, the corticosteroid responsiveness gene signature comprises at least one gene involved in defense response. Examples of defense response genes useful in the methods disclosed herein include KRT6A (e.g., GenBank Accession Nos. NP_005545.1 and NM_005554.3), PROK2 (e.g., GenBank Accession Nos. NP_001119600.1 and NM_001126128.1), SAA4 (e.g., GenBank Accession Nos. NP_006503.2 and NM_006512.3), LYPD1 (e.g., GenBank Accession Nos. NP_001070895.1 and NM_001077427.3), APOBEC3A (e.g., GenBank Accession Nos. NP_001180218.1 and NM_001193289.1), ADAMTS4 (e.g., GenBank Accession Nos. NP_001307265.1 and NM_001320336.1), CD300E (e.g., GenBank Accession NP_852114.2 and NM_181449.2), AGT (e.g., GenBank Accession Nos. NP_000020.1 and NM_000029.3), SAA2-SAA4 (e.g., GenBank Accession Nos. NM_001199744.2 and NP_001186673.1), ITGA2 (e.g., GenBank Accession Nos. NP_002194.2 and NM_002203.3), C2CD4A (e.g., GenBank Accession Nos. NP_001161770.1 and NM_001168298.1), GAL (e.g., GenBank Accession Nos. NP_057057.2 and NM_015973.4), FCN3 (e.g., GenBank Accession Nos. NP_003656.2 and NM_003665.3), FOSL1 (e.g., GenBank Accession Nos. NP_001287773.1 and NM_001300844.1), C4BPA (e.g., GenBank Accession Nos. NP_000706.1 and NM_000715.3), and CD177 (e.g., GenBank Accession No. NM_020406.4 and NP_065139.2).

In some examples, the corticosteroid responsiveness gene signature comprises at least one gene involved response to bacterium genes. Non-limiting examples of genes involved in the response to bacterium to be used as biomarkers in the methods described herein include, DEFB4A (e.g., GenBank Accession Nos. NP_001192195.1 and NM_001205266.1), REG1A (e.g., GenBank Accession Nos. NP_002900.2 and NM_002909.4 3), AQP9 (e.g., GenBank Accession Nos. NP_066190.2 and NM_020980.4), FCGR3B (e.g., GenBank Accession Nos. NP_000561.3 and NM_000570.4), CLEC4D (e.g., GenBank Accession Nos. NP_525126.2 and NM_080387.4), IFNG, TREM1 (e.g., GenBank Accession Nos. NP_001229518.1 and NM_001242589.2), HP (e.g., GenBank Accession Nos. NP_001119574.1 and NM_001126102.2), and PLAU (e.g., GenBank Accession No. NP_001138503.1 and NM_001145031.2). In specific examples, the gene(s) involved in response to bacteria is DEFB4A, FCGR3B, TREM1, or a combination thereof.

In some examples, the corticosteroid responsiveness gene signature comprises at least one gene involved in ion transport and homeostasis biological pathways. Examples include ABCG2 (e.g., GenBank Accession Nos. NP_001244315.1 and NM_001257386.1), SLC26A3 (e.g., GenBank Accession Nos. NP_000102.1 and NM_000111.2), CHP2 (e.g., GenBank Accession Nos. NP_071380.1 and NM_022097.3), CKB (e.g., GenBank Accession Nos. NP_001814.2 and NM_001823.4), KCNJ15 (e.g., GenBank Accession Nos. NP_001263364.1 and NM_001276435.1), SLC6A14 (e.g., GenBank Accession Nos. NP_009162.1 and NM_007231.4), PLLP (e.g., GenBank Accession NP_057077.1 and NM_015993.2), SLC26A2 (e.g., GenBank Accession Nos. NP_000103.2 and NM_000112.3), TRPM6 (e.g., GenBank Accession Nos. NP_001170781.1 and NM_001177310.1), SLC30A10 (e.g., GenBank Accession Nos. NP_061183.2 and NM_018713.2), GLRA2 (e.g., GenBank Accession Nos. NP_001112357.1 and NM_001118885.1), PLA2G12B (e.g., GenBank Accession Nos. NP_001305053.1 and NM_001318124.1), CA1 (e.g., GenBank Accession Nos. NP_001122301.1 and NM_001128829.3), and OTOP2 (e.g., GenBank Accession No. NP_835454.1 and NM_178160.2).

In some examples, the corticosteroid responsiveness gene signature comprises at least one gene involved CXCR1 interaction. Non-limiting examples of genes involved in CXCR1 interaction to be used as biomarkers in the methods described herein include, CSF2, CXCR1 (e.g., GenBank Accession Nos. NP_000625.1 and NM_000634.2), PPBP (e.g., GenBank Accession Nos. NP_002695.1 and NM_002704.3), CXCL6, CMTM2 (e.g., GenBank Accession Nos. NP_001186246.1 and NM_001199317.1), CXCR2, CXCL10 and CXCL11. In specific examples, the gene(s) involved in CXCR1 interaction is CXCR1, CSF2, or a combination thereof.

In some examples, the corticosteroid responsiveness gene signature comprises at least one gene involved in RAGE receptor binding. Examples of RAGE receptor binding genes useful in the methods disclosed herein include, but are not limited to, S100A12 (e.g., GenBank Accession Nos. NP_005612.1 and NM_005621.1), S100A8 (e.g., GenBank Accession Nos. NP_001306126.1 and NM_001319197.1), S100A9 (e.g., GenBank Accession Nos. NP_002956.1 and NM_002965.3), FPR2 (e.g., GenBank Accession Nos. NP_001005738.1 and NM_001005738.1), and FPR1 (e.g., GenBank Accession Nos. NP_001180235.1 and NM_001193306.1). In specific examples, gene(s) involved in RAGE receptor binding for use herein is S100A9.

In some examples, the corticosteroid responsiveness gene signature comprises at least one gene involved in neutrophil deregulation. Non-limiting examples of genes involved in neutrophil degranulation pathways to be used as biomarkers in the methods described herein include, MCEMP1 (e.g., GenBank Accession Nos. NP_777578.2 and NM_174918.2), TNIP3 (e.g., GenBank Accession Nos. NP_001122315.2 and NM_001128843.2), CLEC4D, PPBP, IL11, and CLEC5A (e.g., GenBank Accession Nos. NP_037384.1 and NM_013252.2).

In some examples, the corticosteroid responsiveness gene signature comprises at least one gene involved in granulocyte migration. Examples include CSF3, SAA1 (e.g., GenBank Accession Nos. NP_000322.2 and NM_000331.5), GPR84 (e.g., GenBank Accession Nos. NP_065103.1 and NM_020370.2), SLC11A1 (e.g., GenBank Accession Nos. NP_000569.3 and NM_000578.3), CSF3R (e.g., GenBank Accession Nos. NP_000751.1 and NM_000760.3), PLAU, FCAR (e.g., GenBank Accession Nos. NP_001991.1 and NM_002000.3), and TCN1 (e.g., GenBank Accession Nos. NP_001053.2 and NM_001062.3).

In some examples, the corticosteroid responsiveness gene signature comprises at least one gene involved in endopeptidase activity. Examples include MMP10 (e.g., GenBank Accession Nos. NP_002416.1 and NM_002425.2), MMP1 (e.g., GenBank Accession Nos. NP_002412.1 and NM_002421.3), MMP3 (e.g., GenBank Accession Nos. NP_002413.1 and NM_002422.4), USP2 (e.g., GenBank Accession Nos. NP_001230688.1 and NM_001243759.1), and ADAMTS4

In some examples, the corticosteroid responsiveness gene signature comprises at least one gene involved in peptide cross-linking. Examples include SPRR2A (e.g., GenBank Accession Nos. NP_005979.1 and NM_005988.2), SPRR1B (e.g., GenBank Accession Nos. NP_003116.2 and NM_003125.2), and SPRR3 (e.g., GenBank Accession Nos. NP_001091058.1 and NM_001097589.1).

In some examples, the corticosteroid responsiveness gene signature comprises at least one gene involved in cell adhesion. Examples include CLDN14 (e.g., GenBank Accession Nos. NP_001139549.1 and NM_001146077.1), CLDN1 (e.g., GenBank Accession Nos. NP_066924.1 and NM_021101.4), GLDN (e.g., GenBank Accession Nos. NP_001317226.1 and NM_001330297.1), and IGSF9 (e.g., GenBank Accession Nos. NP_001128522.1 and NM_001135050.1).

In some examples, the corticosteroid responsiveness gene signature comprises at least one gene involved in cyclase activity. Examples include ADGRF1 (e.g., GenBank Accession Nos. NP_079324.2 and NM_025048.3), GUCA2A (e.g., GenBank Accession Nos. NP_291031.2 and NM_033553.2), and GUCA2B (e.g., GenBank Accession Nos. NP_009033.1 and NM_007102.2).

In some examples, the corticosteroid responsiveness gene signature comprises at least one gene involved in lipid metabolic process pathways. Examples include UGT1A8 (e.g., GenBank Accession Nos. NP_061949.3 and NM_019076.4), RBP2 (e.g., GenBank Accession Nos. NP_004155.2 and NM_004164.2), and HMGCS2 (e.g., GenBank Accession Nos. NP_001159579.1 and NM_001166107.1).

In some examples, the corticosteroid responsiveness gene signature comprises at least one gene involved in signaling receptor activity pathways. Examples include HCAR3 (e.g., GenBank Accession Nos. NP_006009.2 and NM_006018.2), and HCAR2 (e.g., GenBank Accession Nos. NP_808219.1 and NM_177551.3).

In some examples, the corticosteroid responsiveness gene signature comprises at least one gene involved in epithelial cell differentiation. Examples include KRT6B (e.g., GenBank Accession Nos. NP_005546.2 and NM_005555.3), and VSIG1 (e.g., GenBank Accession Nos. NP_001164024.1 and NM_001170553.1).

In specific examples, the corticosteroid responsiveness gene signature comprises at least one gene involved in response to bacterium as listed in Table 1, at least one gene involved in CXCR1 interaction or cytokine activity as listed in Table 1, and at least one gene involved in RAGE receptor binding as listed in Table 1. For example, the corticosteroid responsiveness gene signature may comprise at least DEFB4A, CSF2, CXCR1, S100A9, FCGR3B, OSM, TREM1, or a combination thereof. In one specific example, the corticosteroid responsiveness gene signature comprises the combination of DEFB4A, CSF2, CXCR1, S100A9, FCGR3B, OSM, and TREM1.

B. Determination of Corticosteroid Responsiveness Gene Signatures

To determining any of the corticosteroid responsiveness gene signatures as disclosed herein, the expression levels of the genes involved in the corticosteroid responsiveness gene signature in a biological sample of a candidate subject can be measured by routine practice. In some examples, the gene expression levels can be mRNA levels of the target genes. Alternatively, the gene expression levels can be represented by the levels of the gene products (encoded proteins). Assays for measuring levels of mRNA or proteins are known in the art and described herein. See, e.g., Molecular Cloning: A Laboratory Manual, J. Sambrook, et al., eds., Third Edition, Cold Spring Harbor Laboratory Press, Cold Spring Harbor, N.Y., 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York.

A subject to be assessed by any of the methods described herein can be a mammal, e.g., a human patient having UC. A subject having UC may be diagnosed based on clinically available tests and/or an assessment of the pattern of symptoms in a subject and response to therapy. In some embodiments, the subject is a pediatric subject. A pediatric subject may be of 18 years old or below. In some examples, a pediatric patient may have an age range of 0-12 years, e.g., 6 months to 8 years old or 1-6 years. In some instances, the subject may be free of a prior treatment for UC, for example, free of any steroid (e.g., corticosteroid) treatment.

As used herein, the term “biological sample” refers to a sample obtained from a subject. A suitable biological sample can be obtained from a subject as described herein via routine practice. Non-limiting examples of biological samples include fluid samples such as blood (e.g., whole blood, plasma, or serum), urine, and saliva, and solid samples such as tissue (e.g., skin, lung, or nasal) and feces. Such samples may be collected using any method known in the art or described herein, e.g., buccal swab, nasal swab, venipuncture, biopsy, urine collection, or stool collection. In some embodiments, the biological sample can be an intestinal, colon and/or rectal biopsy sample. In one specific example, the biological sample is a rectal tissue sample.

The expression level(s) of the genes involved in any of the corticosteroid responsiveness signature as disclosed herein may be represented by the level of the mRNAs. Methods for detecting and/or assessing a level of nucleic acid expression in a sample are well known in the art, and all suitable methods for detecting and/or assessing an amount of nucleic acid expression known to one of skill in the art are contemplated within the scope of the invention. Non-limiting examples of suitable methods to assess an amount of nucleic acid expression may include arrays, such as microarrays, PCR, such as RT-PCR (including quantitative RT-PCR), nuclease protection assays and Northern blot analyses.

The level of expression of the target genes may be normalized to the level of a control nucleic acid. This allows comparisons between assays that are performed on different occasions. For example, the raw data of gene expression levels can be normalized against the expression level of an internal control RNA (e.g., a ribosomal RNA or U6 RNA). The normalized expression level(s) of the genes can then be compared to the expression level(s) of the same genes of a control tissue sample, which can be normalized against the same internal control RNA, to determine whether the subject is likely to be responsive to a therapeutic treatment or non-responsive to a therapeutic treatment.

In another embodiment, the levels of the genes can be determined by measuring the gene products at the protein level in a biological sample. In a specific embodiment, protein expression may be measured using an ELISA to determine the expression level of the genes involved in the corticosteroid responsiveness gene signature as disclosed herein in a biological sample as also disclosed herein. Methods for detecting and/or assessing an amount of protein expression are well known in the art, and all suitable methods for detecting and/or assessing an amount of protein expression known to one of skill in the art are contemplated within the scope of the invention. Non-limiting examples of suitable methods to detect and/or assess an amount of protein expression may include epitope binding agent-based methods and mass spectrometry based methods.

Based on the expression levels of the involved genes disclosed herein, a corticosteroid responsiveness gene signature can be obtained via, e.g., a computational program. Various computational programs can be applied in the methods of this disclosure to aid in analysis of the expression data for producing the gene signature. Examples include, but are not limited to, Prediction Analysis of Microarray (PAM; see Tibshirani et al., PNAS 99(10):6567-6572, 2002); Plausible Neural Network (PNN; see, e.g., U.S. Pat. No. 7,287,014), PNNSulotion software and others provided by PNN Technologies Inc., Woodbridge, Va., USA, and Significance Analysis of Microarray (SAM). In some examples, a gene signature may be represented by a score that characterizes the expression pattern of the genes involved in the gene signature. See also Examples below.

C. Assessing Steroid Responsiveness Based on Corticosteroid Responsiveness Gene Signature and Optionally Other Factors

Any of the corticosteroid responsiveness gene signature of a candidate subject as disclosed herein can be used for assessing whether the subject's responsiveness or non-responsiveness to a UC therapy, for example, a steroid therapy (e.g., a corticosteroid therapy, an anti-TNFa therapy, or an anti-α4β7 integrin therapy). For example, the corticosteroid responsiveness gene signature of a candidate subject can be compared with a pre-determined value.

A pre-determined value may represent the same corticosteroid responsiveness gene signature of a control subject or represent the same gene signature of a control population. In some examples, the same gene signature of a control subject or a control population may be determined by the same method as used for determining the gene signature of the candidate subject. In some instances, the control subject or control population may refer to a healthy subject or healthy subject population of the same species (e.g., a human subject or human subject population having no UC). Alternatively, the control subject or control population may be a UC patient or UC patient population who is responsive to any of the therapeutic agents disclosed herein. In other instances, the control subject or control population may be a UC patient or UC patient population who is non-responsive to the therapeutic agent.

It is to be understood that the methods provided herein do not require that a pre-determined value be measured every time a candidate subject is tested. Rather, in some embodiments, it is contemplated that the pre-determined value can be obtained and recorded and that any test level can be compared to such a pre-determined level. The pre-determined level may be a single-cutoff value or a range of values.

By comparing the corticosteroid responsiveness gene signature of a candidate subject as disclosed herein and a pre-determined value as also described herein, the subject can be identified as responsive or likely to be responsive or as not responsive or not likely to be responsive to steroid treatment based on the assessing.

For example, when the pre-determined value represents the same gene signature of UC patients who are responsive to a therapy, derivation from such a pre-determined value would indicate non-responsiveness to the therapy. Alternatively, when the pre-determined value represents the same gene signature of UC patients who are non-responsive to a therapy, derivation from such a pre-determined value would indicate responsiveness to the therapy. In some instances, derivation means that the gene signature (e.g., represented by a score) of a candidate subject is elevated or reduced as relative to a pre-determined value, for example, by at least 5%, 10%, 20%, 30%, 40%, 50%, 60%, 70%, 80%, 90%, 100%, 150%, 200%, 300%, 400%, 500% or more above or below the pre-determined value.

In addition to the corticosteroid responsiveness gene signature, a subject's responsive or non-responsiveness to the treatment disclosed herein may further take into consideration one or more clinical factors. Exemplary clinical factors include, but are not limited to, gender, levels of rectal eosinophils, and/or disease severity. In some examples, levels of rectal eosinophils may be represented by the expression level of ALOX15. In that case, any of the methods disclosed herein may further comprise measuring the expression level of ALOX15 in a biological sample (e.g., a rectal biopsy sample) of the candidate subject.

Alternatively or in addition, assessing responsiveness or non-responsiveness of a subject may further comprise factors such as microbial populations in the biological sample, such as rectal biopsy of the subject. In that case, any of the methods disclosed herein may further comprise analyzing microbial populations in the biological sample. Microbial populations can be determined using methods well known in the art, including, for example, 16S RNA gene sequencing. Ribosomal RNA genes from a biological samples, microcolonies or cultures from a subject having UC can be amplified by PCR by using specific 16S RNA oligonucleotide primers for bacteria. After cloning the PCR products, the inserts are screened by their restriction patterns (RFLP—restriction fragment length polymorphism). The clones can be submitted to sequence analysis and compared with known 16S RNA genes using, for example, the online GenBank database. In this way, it can be determined which microorganism species are present or absent. Associations between disease severity associated taxa such as Campylobacter, Veillonella, and Enterococcus with genes and pathways linked to a more severe disease form, and refractory disease in connection with initial corticosteroid induction therapy. In contrast, decreased taxa from the Clostridiales order that are considered beneficial, which show a negative correlation with gene signatures associated with disease severity and unfavorable treatment responses. Accordingly, presence of a microbial population associated with disease severity would be indicative of non-responsiveness to the treatment, while presence of a beneficial microbial population would be indicative of responsiveness to the treatment.

II. Assessment of UC Disease Occurrence and/or Severity

Another aspect of the present disclosure relates to methods for identifying a subject having or at risk for UC, or for determining disease severity of a UC patient (e.g., whether the patient has active disease), based on the UC occurrence and/or severity gene signature as disclosed herein. The UC occurrence and/or severity gene signature may comprise one or more genes involved in mitochondrial function, one or more genes involved in the Kreb cycle, or a combination thereof.

In some examples, the UC disease occurrence or severity gene signature may comprise at least one gene involved in mitochondrial function. Examples of mitochondrial function genes useful in the methods disclosed herein include, PPARGC1A (PCG-1α) (e.g., GenBank Accession Nos. NP_001317680.1 and NM_001330751.1), MT-COL COX5A (e.g., GenBank Accession Nos. NP_004246.2 and NM_004255.3), a Complex 1 gene, a Complex II gene, a Complex II gene, a Complex IV gene, a Complex V gene, or a combination thereof. Non-limiting examples of a Complex I gene include, MT-ND1 (e.g., GenBank Accession Nos. YP_003024026.1 and NC_012920.1), MT-ND2 (e.g., GenBank Accession Nos. YP_003024027.1 and NC_012920.1), MT-ND3 (e.g., GenBank Accession Nos. YP_003024033.1 and NC_012920.1), MT-ND4 (e.g., GenBank Accession Nos. YP_003024035.1 and NC_012920.1), MT-ND4L (e.g., GenBank Accession Nos. YP_003024034.1 and NC_012920.1), MT-ND5 (e.g., GenBank Accession Nos. YP_003024036.1 and NC_012920.1), and MT-ND6 (e.g., GenBank Accession Nos. YP_003024037.1 and NC_012920.1). Non-limiting examples of a Complex III gene include, MT-CYB (e.g., GenBank Accession Nos. YP_003024038.1 and NC_012920.1). Non-limiting examples of a Complex IV gene include, MT-CO1 (e.g., GenBank Accession Nos. YP_003024028.1 and NC_012920.1), MT-CO2 (e.g., GenBank Accession Nos. YP_003024029.1 and NC_012920.1), and MT-CO3 (e.g., GenBank Accession Nos. YP_003024032.1 and NC_012920.1). Non-limiting examples of a Complex V gene include, MT-ATP6 (e.g., GenBank Accession Nos. YP_003024031.1 and NC_012920.1) and MT-ATP8 (e.g., GenBank Accession Nos. YP_003024030.1 and NC_012920.1). In some examples, the gene involved in mitochondrial function comprises PPARGC1A (PCG-1α). Alternatively or in addition, the gene involved in mitochondrial function comprises MT-CO1 and/or COX5A, for example, MT-CO1⁺ and/or COX5A⁺ cells.

In some examples, the UC disease occurrence or severity gene signature may comprise at least one gene involved in the Kreb cycle. Examples of genes involved in the Kreb cycle (TCA cycle) useful in the methods disclosed herein include, but are not limited to, ACO2 (e.g., GenBank Accession Nos. NP_001089.1 and NM_001098.2), BSG (e.g., GenBank Accession Nos. NP_001309172.1 and NM_001322243.1), COX5B (e.g., GenBank Accession Nos. NP_001853.2 and NM_001862.2), COX6C (e.g., GenBank Accession Nos. NP_004365.1 and NM_004374.3), CYC1 (e.g., GenBank Accession Nos. NP_001907.2 and NM_001916.4), CYCS (e.g., GenBank Accession Nos. NP_061820.1 and NM_018947.5), DLD (e.g., GenBank Accession Nos. NP_000099.2 and NM_000108.4), ETFA (e.g., GenBank Accession Nos. NP_000117.1 and NM_000126.3), ETFDH (e.g., GenBank Accession Nos. NP_001268666.1 and NM_001281737.1), MPC2 (e.g., GenBank Accession Nos. NP_001137146.1 and NM_001143674.3), NDUFA2 (e.g., GenBank Accession Nos. NP_001171941.1 and NM_001185012.1), NDUFA5 (e.g., GenBank Accession Nos. NP_001269348.1 and NM_001282419.2), NDUFA6 (e.g., GenBank Accession Nos. NP_002481.2 and NM_002490.4), NDUFB10 (e.g., GenBank Accession Nos. NP_004539.1 and NM_004548.2), NDUFB5 (e.g., GenBank Accession Nos. NP_001186886.1 and NM_001199957.1), NDUFB9 (e.g., GenBank Accession Nos. NP_001298097.1 and NM_001311168.1), NDUFS1 (e.g., GenBank Accession Nos. NP_001186910.1 and NM_001199981.1), NNT (e.g., GenBank Accession Nos. NP_036475.3 and NM_012343.3), NUBPL (e.g., GenBank Accession Nos. NP_001188502.1 and NM_001201573.1), PDHA1 (e.g., GenBank Accession Nos. NP_000275.1 and NM_000284.3), PDK2 (e.g., GenBank Accession Nos. NP_001186827.1 and NM_001199898.1), PDK4 (e.g., GenBank Accession Nos. NP_002603.1 and NM_002612.3), SDHB (e.g., GenBank Accession Nos. NP_002991.2 and NM_003000.2), SDHD (e.g., GenBank Accession Nos. NP_001263432.1 and NM_001276503.1), SLC16A1 (e.g., GenBank Accession Nos. NP_001159968.1 and NM_001166496.1), SUCLG1 (e.g., GenBank Accession Nos. NP_001159968.1 and NM_001166496.1), and SUCLG2 (e.g., GenBank Accession Nos. NP_001171070.1 and NM_001177599.1). The UC disease occurrence and/or severity gene signature may comprise at least 2 genes, at least 3 genes, at least 4 genes, at least 5 genes, at least 6 genes, at least 7 genes, at least 8 genes, at least 9 genes, at least 10 genes, or at least 15 genes selected from the above list. In specific examples, the UC disease occurrence and/or severity gene signature consists of all of the Kreb cycle genes listed above.

In some examples, the UC disease occurrence or severity gene signature may comprise gene involved the Kreb cycle, which may be COX5B, COX6C, NDUFA2, NDUFA5, NDUFA6, NDUFB10, NDUFB5, NDUFB9, NDUFS1, SLC16A1, or a combination thereof. In specific examples, the UC disease occurrence and/or severity gene signature may comprise all of COX5B, COX6C, NDUFA2, NDUFA5, NDUFA6, NDUFB10, NDUFB5, NDUFB9, NDUFS1, and SLC16A1.

The expression level(s) of the genes involved in any of the UC occurrence nd/or disease severity gene signatures as disclosed herein may be represented by the level of the mRNAs. Alternatively, the expression level(s) of the genes may be represented by the level(s) of the gene product, including, for example, cell-surface expressed gene product. Methods for measuring mRNA or proteins levels are well-known in the art. See also disclosures above.

Based on the expression levels of the involved genes disclosed herein, a UC occurrence and/or disease severity gene signature can be obtained via, e.g., a computational program, such as those disclosed herein. In some instances, the UC occurrence and/or disease severity gene signature may be represented by a score as calculated by the computational program.

Any of the UC occurrence and/or disease severity gene signatures of a candidate subject as disclosed herein can be used for assessing whether the subject has or is at risk for US. In some instances, such a gene signature may be used in determining whether a UC patient has active disease. For example, the UC occurrence and/or disease severity gene signature of a candidate subject can be compared with a pre-determined value, which may represent the same gene signature of a control subject or represent the same gene signature of a control population. In some examples, the same gene signature of a control subject or a control population may be determined by the same method as used for determining the gene signature of the candidate subject. In some instances, the control subject or control population may refer to a healthy subject or healthy subject population of the same species (e.g., a human subject or human subject population having no UC). Alternatively, the control subject or control population may be a UC patient or UC patient population who has inactive disease. In other instances, the control subject or control population may be a UC patient or UC patient population who has active disease.

It is to be understood that the methods provided herein do not require that a pre-determined value be measured every time a candidate subject is tested. Rather, in some embodiments, it is contemplated that the pre-determined value can be obtained and recorded and that any test level can be compared to such a pre-determined level. The pre-determined level may be a single-cutoff value or a range of values.

By comparing the UC occurrence and/or disease severity gene signature of a candidate subject as disclosed herein and a pre-determined value as also described herein, the subject can be identified as having or at risk for the disease, or having active disease.

For example, when the pre-determined value represents the same gene signature of healthy controls, derivation from such a pre-determined value would indicate disease occurrence of risk for the disease. Alternatively, when the pre-determined value represents the same gene signature of UC patients in inactive disease state, derivation from such a pre-determined value would indicate active disease.

UC disease severity the severity of UC can be graded through clinical examination, for example, a mild UC grade is indicated by bleeding per rectum and fewer than four bowel motions per day; a moderate UC grade is indicated by bleeding per rectum with more than four bowel motions per day; and severe UC grade is indicated by bleeding per rectum, more than four bowel motions per day, and a systemic illness with hypoalbuminemia (<30 g/L).

### III. Therapeutic Application of UC Gene Signatures

When a subject is determined to be responsive or non-responsive based on any of the corticosteroid responsiveness gene signatures disclosed herein, this subject could be subjected to a suitable treatment for UC, including any of the UC treatments known in the art and disclosed herein. Alternatively, when a subject is determined as having or at risk for US or having active disease based on any of the UC occurrence and/or disease severity gene signatures as also disclosed herein, such a subject may be given a suitable anti-UC therapy, for example, those described herein.

In some embodiments, a subject is determined to be likely responsive to a steroid therapy, an anti-TNFα therapy, or an anti-α4β7 integrin therapy, using any of the methods described herein, the subject may then be administered an effective amount of a steroid, an anti-TNFα agent, and/or an anti-anti-α4β7 integrin agent, for treating UC. In some examples, such a subject may be given a steroid compound, such as a corticosteroid compound.

In some embodiments, a subject is determined to be unlikely responsive to a steroid therapy, an anti-TNFα therapy, or an anti-α4β7 integrin therapy, using any of the methods described herein, the subject may then be administered an effective amount of an alternative therapeutic agent for treating UC, for example, a non-steroid, a non-anti-TNFα agent, and/or non-anti-anti-α4β7 integrin agent.

In some embodiments, a subject is determined to have or at risk for UC and can be can be treated by a suitable anti-UC therapy, such as those described herein. Alternatively, a subject is determined to have active disease of UC and can be treated by a suitable anti-UC therapy or subject to adjustment of current therapy (e.g., switch to a different therapeutic agent or adjust treatment conditions such as doses or dosing schedules of the current therapeutic agent).

Non-limiting examples of steroids include corticosteroids such as methylprednisolone, prednisone, hydrocortisone, and budesonide. In another aspect, a subject determined to be likely responsive using the methods described herein, may be administered an effective amount of an anti-TNF therapy for treating UC.

Non-limiting examples of Tumor Necrosis Factor Inhibitors include Infliximab, Golimuab, and Adalimumab. In yet another aspect, a subject determined to be likely responsive using the methods described herein, may be administered an effective amount of an anti-integrin α4β7 therapy (e.g., Vedolizumab) for treating UC. In some embodiments a subject determined to be likely responsive using the methods described herein may be administered a steroid, anti-TNF and/or anti-integrin α4β7 therapy in addition to any of the UC treatments known in the art.

For example, medications such as sulfasalazine (Azulfadine), mesalamine (Asacol, Pentasa), azathioprine (Imuran), 6-MP (Purinethol), cyclosporine, and methotrexate, can be administered to the subject in an amount effective to treating UC. In some embodiments, the UC treatment comprises an anti-inflammatory agent, an immune suppressant agent, an antibiotic agent, or a combination thereof. Non-limiting examples of anti-inflammatory agents include sulfasalazine, mesalamine, balsalazide, olsalazine, or corticosteroids (e.g., prednisone or budesonide). Non-limiting examples of immune suppressant agents include azathioprine, mercaptopurine, cyclosporine, infliximab, adalimumab, certolizumab pegol, methotrexate, or natalizumab. Non-limiting examples of antibiotics include metronidazole and ciprofloxacin. In some embodiments, UC treatment comprises an anti-diarrheal (e.g., psyllium powder, methylcellulose or loperamide), a laxative, acetaminophen, iron, vitamin B-12, calcium, or vitamin D. In some embodiments, UC treatment comprises surgery or fecal bacteriotherapy (also called a fecal microbiota transplantation or stool transplant).

Non-limiting examples of surgery include proctocolectomy, ileostomy, or strictureplasty. In some embodiments, UC treatment comprises a therapeutic agent (e.g., an anti-inflammatory agent, an immune suppressant agent, an antibiotic agent, or a combination thereof) and surgery. It is to be understood that any of the UC treatments described herein may be used in any combination. According to the method disclosed herein, a subject determined to be non-responsive to a therapeutic agent may be administered a non-steroid, non-anti-TNF, and non-anti-integrin α4β7 therapy for treating UC

The term “treating” as used herein refers to the application or administration of a composition including one or more active agents to a subject, who has UC, a symptom of UC, or a predisposition toward UC, with the purpose to cure, heal, alleviate, relieve, alter, remedy, ameliorate, improve, or affect the disease, the symptoms of the disease, or the predisposition toward the disease. An “effective amount” is that amount of an anti-UC agent that alone, or together with further doses, produces the desired response, e.g. eliminate or alleviate symptoms, prevent or reduce the risk of flare-ups (maintain long-term remission), and/or restore quality of life. The desired response is to inhibit the progression of the disease. This may involve only slowing the progression of the disease temporarily, although more preferably, it involves halting the progression of the disease permanently. This can be monitored by routine methods or can be monitored according to diagnostic and prognostic methods discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Any of the methods described herein can further comprise adjusting the UC treatment performed to the subject based on the results obtained from the methods disclosed herein (e.g., based on gene signatures disclosed herein). Adjusting treatment includes, but are not limited to, changing the dose and/or administration of the anti-UC agent used in the current treatment, switching the current medication to a different anti-UC agent, or applying a new UC therapy to the subject, which can be either in combination with the current therapy or replacing the current therapy.

In some embodiments, the present disclosure provides a method for treating a subject (e.g., a human patient) having ulcerative colitis (UC), the method comprising administering an effective amount of an anti-UC agent (e.g., those disclosed herein) to a subject who exhibits a gene signature indicative of responsiveness or non-responsiveness to a steroid therapy, an anti-TNFa therapy, and/or an anti-α4β7 integrin therapy. If the subject is predicted as responsiveness to the therapy based on the corresponding gene signature as disclosed herein, the same therapy can be applied to the subject. Alternatively, if the subject is predicted as not responsiveness to the therapy based on the corresponding gene signature, a different type of therapy (e.g., a non-steroid therapy) can be applied to the subject.

In some embodiments, the present disclosure provides a method for treating a subject (e.g., a human patient) having or at risk for UC, or having active UC, the method comprising administering an effective amount of an anti-UC agent (e.g., those disclosed herein) to a subject who exhibits a gene signature indicative of disease occurrence and/or disease severity.

### IV. Kits for Use in Assessing UC Gene Signatures and UC Therapy

Also within the scope of this disclosure are kits for use in assessing responsiveness to a UC therapy in a subject, such as a human subject. Such a kit can comprise reagents for determining the level(s) of genes involved in any of the corticosteroid responsiveness gene signature (see Table 1), or genes involved in any of the UC occurrence and/or disease severity gene signatures as disclosed herein. The reagents can be oligonucleotide probes/primers for determining the mRNA levels of the target genes. Alternatively, the kit can contain antibodies specific to one or more of these gene products. In specific examples, the kit comprises reagents for determining the levels of one or more of DEFB4A, CSF2, CXCR1, S100A9, FCGR3B, OSM, and TREM1.

Any of the kits described herein can further comprise an instruction manual providing guidance for using the kit to perform the diagnostic/prognostic methods.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Ulcerative Colitis Mucosal Transcriptomes Reveal Mitochondriopathy and Personalized Mechanisms Underlying Disease Severity and Treatment Response

The goal of this study was to gain a greater understanding of individualized pathways driving clinical and mucosal severity and response to therapy in ulcerative colitis by applying a standardized approach to a large, multicenter inception cohort that collected samples before treatment initiation, and included subjects representing the full spectrum of disease severities.

Here, RNA-seq analysis was performed to define pre-treatment rectal gene expression, and fecal microbiota profiles, in 206 pediatric ulcerative colitis (UC) patients receiving standardized therapy. Key findings in adult and pediatric UC cohorts of 408 participants were validated in this study. It was observed that a marked suppression of mitochondrial genes and function across cohorts in active UC, and that increasing disease severity is notable for enrichment of adenoma/adenocarcinoma and innate immune genes. A subset of severity genes improves prediction of corticosteroid-induced remission in the discovery cohort. This gene signature is also associated with response to anti-TNFα and anti-α4β7 integrin in adult cohorts. The severity and therapeutic responsiveness gene signatures were in turn associated with shifts in microbes previously implicated in mucosal homeostasis.

Taken together, the instant study has captured robust gene expression and pathways that are linked to UC pathogenesis, severity, response to corticosteroid therapy, and gut microbiota. The results reported herein provide new insights into molecular mechanisms driving disease course.

### Methods

### Study Design and Participants

Predicting Response to Standardized Pediatric Colitis Therapy (PROTECT) was a multicenter inception cohort study based at 29 centers in the USA and Canada. Children aged 4-17 years with a diagnosis of UC based on accepted clinical, endoscopic, and histological parameters, disease extent beyond the rectum, a baseline Pediatric Ulcerative Colitis Activity Index (PUCAI) score of at least 10, no previous therapy for colitis, and stool culture negative for enteric bacterial pathogens and Clostridium difficile toxin were included. Detailed protocol and study description can be found in Hyams et al., Lancet Gastroenterol Hepatol, doi:10.1016/52468-1253(17)30252-2 (2017) and Hyams et al., The Journal of pediatrics 129, 81-88, (1996). Disease extent was classified as proctosigmoiditis, left-sided colitis (to the splenic flexure), extensive colitis (to the hepatic flexure), or pancolitis (beyond the hepatic flexure) by visual evidence. Patients with severe or fulminant disease at presentation who received a flexible sigmoidoscopy because of safety concerns were assigned to the extensive colitis group (unassessable). Clinical activity at diagnosis was established with the PUCAI (range 0-85), Mayo endoscopic scope (grade 1-3), and total Mayo score (range 0-12). PUCAI less than 10 denoted inactive disease or remission, 10-30 denoted mild disease, 35-60 denoted moderate disease, and 65 or higher denoted severe disease. A central pathologist blinded to clinical data examined a single rectal biopsy from each patient and assessed histological features of chronicity and quantitated acute inflammation. Paneth cell metaplasia, surface villiform changes, or basal lymphoid aggregates were recorded if present. The description of eosinophilic inflammation included the peak number of eosinophils per high-power field relative to a cut-point (>32 cells per high-power field) derived from a study of normal rectal biopsies in children.

Depending on initial PUCAI score, patients received initial treatment with either mesalamine (mild disease), or corticosteroids (moderate and severe disease), with some physician discretion allowed. A detailed description of treatment guidelines is provided in Hyams et al., Lancet Gastroenterol Hepatol, doi:10.1016/52468-1253(17)30252-2 (2017) and Hyams et al., The Journal of pediatrics 129, 81-88, (1996). All patients on mesalamine received study-supplied Pentasa (Shire Pharmaceuticals/Pantheon, Greenville, N.C., USA). For this part of the study, a week 4 (W4) remission outcome defined as PUCAI<10 was used without additional therapy or colectomy. Twenty additional patients were enrolled and were included in the current analyses as non-IBD controls after clinical endoscopic, and biopsies evaluation demonstrated no histologic and endoscopic inflammation. Rectal mucosal biopsies from a representative sub-cohort of 206 PROTECT UC patients and 20 age and gender matched non-IBD controls underwent high coverage transcriptomic profiling using Illumina RNAseq (see Table 2 below). These constituted the Discovery cohort for the current study.

The representative sub cohort for RNAseq was defined by having a baseline rectal biopsy available to be included in the RNA seq analysis, and must also have the following data available in order to be assigned to the appropriate clinical subgroup: baseline PUCAI, medication data including the need for rescue or colectomy through week 4 and a week 4 PUCAI if the participant has not required rescue or a colectomy during the first four weeks. The following PROTECT participants were not eligible for the RNA seq analysis: patients with a diagnosis other than UC after enrollment, patients with significant baseline violations, patients who took rescue medications for a non-UC reason within the first four weeks, baseline RNA sample is unavailable, race is either ‘Asian’, ‘Black or African American’ or ‘Unknown’, baseline PUCAI<35 but did not start on mesalamine as first therapy, baseline PUCAI>=35 but did not start on corticosteroids as first therapy. A total of 219 were selected, and data for 206 were ultimately available, after excluding 5 subjects based on the RNAseq data as described below, and 8 with insufficient RNA.

TABLE 2
Characteristics of Controls and PROTECT Ulcerative Colitis Discovery and Validation Cohorts.

| | | | UC | | | | | |
|---|---|---|---|---|---|---|---|---|
| | Ctl | | (n = 428) | | UC | | UC mild | |
| | (n = 20) | | Full PROTECT | | (n = 206) | | (n = 54) | |
| | RNAseq | | Cohort | | RNAseq | | RNAseq | |
| Age (Mean ± SD) | 13.9 ± 3.3 | | 12.7 ± 3.3 | | 12.9 ± 3.2 | | 13.1 ± 3.5 | |
| Sex M (%) | 9 | (45%) | 216 | (50%) | 112 | (54%) | 32 | (59%) |
| BMI z score (Mean ± SD) | 0.3 ± 1.6 | | −0.2 ± 1.3 | | −0.26 ± 1.32 | | −0.08 ± 1.19 | |
| White | 17/20 | (85%) | 351/420 | (84%) | 204/206 | (99%) | 52/54 | (96%) |

| PUCAI score (range 0-85) | | | | | | | |
|---|---|---|---|---|---|---|---|
| 10-30 (Mild) | — | 102 | (24%) | 54 | (26%) | 54 | (100% |
| 35-60 (Moderate) | — | 185 | (43%) | 84 | (41%) | — | |
| ≥65 (Severe) | — | 141 | (33%) | 68 | (33%) | — | |
| Mayo endoscopy subscore | | | | | | | |
| (range 0-3) | | | | | | | |
| Grade 1 Mild | — | 59 | (14%) | 27 | (13%) | 20 | (37%) |
| Grade 2 Moderate | — | 224 | (52%) | 108 | (52%) | 29 | (54%) |
| Grade 3 Severe | — | 145 | (34%) | 71 | (34%) | 5 | (9%) |
| Disease location | | | | | | | |
| Proctosigmoiditis | — | 29 | (7%) | 14 | (7%) | 11 | (20%) |
| Left-sided colitis | — | 44 | (10%) | 25 | (12%) | 14 | (26%) |
| Extensive/Pancolitis/ | — | 355 | (83%) | 167 | (81%) | 29 | (54%) |
| *Unassessable | | | | | | | |
| Initial Treatment | | | | | | | |
| Mesalamine | — | 136 | (32%) | 53 | (26%) | 53 | (98%) |
| Oral or IV steroids | — | 292 | (68%) | 153 | (74%) | 1 | (2%) |
| Oral steroids | — | 144 | (34%) | 82 | (40%) | 1 | (2%) |
| IV steroids | — | 148 | (34%) | 71 | (34%) | — | |
| Week 4 remission (PUCAI <10) | — | 211/422 | (50)% | 105 | (51%) | 30 | (56%) |
| Week 4 fecal calpro <250 | — | 56/282 | (20%) | 39/150 | (26%) | 14/42 | (33%) |

*Unassessable: severe/fulminant disease at presentation and the clinician performed a flexible sigmoidoscopy for safety concerns.
Data are mean ± SD, n (%), n/N (%) unless noted otherwise.
n/N values show missing data.
PUCAI = Pediatric Ulcerative Colitis Activity Index.

### Rectal RNA Extraction and RNA-Seq Analysis

RNA was isolated from rectal biopsies obtained during diagnostic colonoscopy using the Qiagen AllPrep RNA/DNA Mini Kit. PolyA-RNA selection, fragmentation, cDNA synthesis, adaptor ligation, TruSeq RNA sample library preparation (Illumina, San Diego, Calif.), and paired-end 75 bp sequencing was performed. An additional validation of the baseline rectal gene expression at diagnosis utilized the independent RISK cohort of treatment naïve pediatric patients (55 non-IBD controls, 43 UC patients, and 92 CD patients with rectal inflammation) and single-end 75 bp mRNA sequencing was performed. Reads were quantified by kallisto, using Gencode v24 as the reference genome and Transcripts per Million (TPM) as an output. We included 14,085 protein-coding mRNA genes with TPM above 1 in 20% of the samples in our downstream analysis. Only samples for which the gene expression (Y encoded genes and XIST) determined gender matched the clinical reported gender were included in the analyses (we excluded only 1 sample with unmatched gender). Four other PROTECT samples were excluded due to poor read quality. A total of 226 RNAseq samples with mean read depth of ˜47M (14M Std. Deviation) were stratified into specific clinical sub-groups including Ctl (n=20), and UC (n=206), and were sub-stratified based on disease severity, and on histologic findings. Differentially expressed genes were determined in GeneSpring® software with fold change differences (FC)>=1.5 and using the Benjamini-Hochberg false discovery rate correction (FDR, 0.001) for all analyses except for the corticosteroid response genes that was calculated out of the 712 severity genes with FDR<0.05. Unsupervised hierarchical clustering using Euclidean distance metric and Ward's linkage rule was used to test for groups of rectal biopsies with similar patterns of gene expression. ToppGene and ToppCluster software were used to test for functional annotation enrichment analyses of immune cell types, pathways, phenotype, immune cell type enrichments, and biologic functions. Visualization of the network was obtained using Cytoscape.v3.0.2 52.

For validation of the association between baseline gene expression and outcome, independent Lexogen QuantSeq 3′ mRNA-Seq libraries were generated and single-end 100 bp sequencing was performed for 134 participants who also had Illumina mRNA-Seq data (the Discovery Cohort) and for 50 participants who did not have Illumina mRNA-Seq data (the independent Validation cohort; see Table 1 above). Principal Coordinates Analysis (PCA) was performed to summarize variation in gene expression between patients, and principal components (PC) values were extracted for downstream analyses. The following were taken into consideration: (i) several central gene expression pathways PC1 pre-identified by the previous differential expression analyses, and (ii) functional annotation enrichment analyses of the core 5296 UC genes, the 712 severity genes, and the 115 corticosteroid responsiveness gene signature for the model building and associations with the microbial composition as described below. PROTECT (GSE109142) and RISK (GSE117993) rectal mRNAseq data sets were deposited into GEO.

### Analyses of Microarrays

Colon biopsy gene expression data and patient clinical data from published studies available in Gene Expression Omnibus (GEO) were obtained. The Affymetrix raw gene array data (.CEL files) were processed to obtain a log 2 expression value for each gene probe set using the robust multichip average (RMA) method implemented in R; the Affymetrix GeneChip Human Genome U133 Plus 2.0 Arrays were processed in R with the affy package (v1.56.0) and the gcrma package (2.50.0), and the Human Gene 1.0 ST arrays were processed with the oligo package (v1.42.0). For comparative analysis, the LIMMA package was used to identify the filtered gene probe sets that showed significant differential expression between the studied groups, based on moderated t-statistics with Benjamini-Hochberg false discovery rate (FDR) correction for multiple testing. Gene probe sets were selected as biologically significant using FDR<0.05 and a fold change (FC)≥1.5. When genes in microarray data were represented by multiple probes, the probe with the greatest interquartile range was selected for analysis. PCA was performed on the normalized log 2 microarray data of control and UC samples and PC1 values were calculated.

### Microbiome Analyses

DNA was extracted from PROTECT UC stool samples and subjected to 16S rRNA amplicon sequencing. Operational Taxonomic Unit (OTU) clustering and taxonomic assignment was performed 24 (NCBI SRA Bioproject: PRJNA436359). Briefly, for the OTU analysis the 16S bioBakery workflow built with AnADAMA2 was applied and microbial taxonomy was based on the Greengenes 16S rDNA database (version 13.5). Samples were subsequently filtered (min 3,000 reads and OTU prevalence threshold of 20 samples). Statistical significance was established using hierarchical all-against-all association testing (HAllA) in all-against-all mode using Spearman as the similarity measure and a cut-off of 0.2 for the false discovery rate. Overall, 156 PROTECT stools at baseline were available that also had mRNAseq data. In total, 149 OTUs were significantly associated with 9 genes, and 15 pathways, with 36 below FDR 0.1. Overall, only 28 RISK CD cases and 21 PROTECT Lexogen UC validation cohort cases had both fecal microbial profiling and rectal mRNAseq data, providing insufficient power for validation of these results.

### Computational Deconvolution

To estimate cell subset proportions, a cell-type deconvolution was performed. xCell 56, a computational method that is able to infer 64 various cell types (e.g., immune cell types, epithelial, and stroma cell types) using gene signatures, was used. To ensure robustness of our downstream analyses, only cell types that had significant enrichment scores (FDR corrected p-values <0.1 in at least 80% of the samples) were considered. The significance was calculated using two approaches, taking into account cell types that were significant in at least one of them. The first includes randomization of the genes in the signatures used for generating the enrichment scores and the second includes using simulations where the tested cell type is not included in the mixture. Epithelial cells were considered but did not vary significantly between samples. The following significant cell types were identified: active Dendritic Cells, Astrocytes, B-cells, CD4+ naive T-cells, Conventional dendritic cells, Dendritic Cells, Memory B-cells, Plasma cells, Th1 cells, and Monocytes. The scores of active Dendritic Cells and Dendritic cells as well as B-cells and “Memory B-cells” across samples were positively and highly correlated and we consider the more specific and biologically relevant activated DC and Memory B-cells. Astrocytes cell type was removed from the calculation.

### High-Resolution Respirometry

The Oxygraph-2k (O2k, Oroboros Instrutments, Innsbruck, Austria) was used for measurements of respiration. Each chamber was air-calibrated in Mir05 respiration medium (0.5 mM EDTA, 3 mM MgCl₂, 60 mM k-lactobionic acid, 20 mM taurine, 10 mM KH₂PO₄, 20 mM HEPES, 110 mM D-sucrose, 0.1% BSA essentially fatty acid free) before each experiment. All experiments were performed at 37° C. Oxygen concentrations in each chamber never dropped below 80 uM during any experiment. Patient biopsies were taken from the cecum and rectum in both control patients (N=5) and patients with ulcerative colitis (N=9). Cecal and rectal biopsies were homogenized in Mir05 respiration medium, and 100 μl of the tissue homogenate was added to each chamber. Once baseline oxygen levels in each chamber became stable, cytochrome c (10 μM), malate (2 mM), pyruvate (5 mM), ADP (5 mM), and glutamate (10 mM) were added to stimulate respiration through Complex I. Once the oxygen consumption rate plateaued, succinate (10 mM) was added to assess the combined activity of Complexes I+II. Next, rotenone (1 mM) was added to inhibit Complex I activity, and additional succinate was added to analyze maximal Complex II activity. Carbonyl cyanide p-trifluoromethoxyphenylhydrazone (FCCP; 0.5 μM) was then added to uncouple the mitochondrial membrane and induce maximal respiration. Respiration rates were normalized to the amount of protein added for each sample. Complex I respiration was defined as the rate of respiration of malate/ADP/pyruvate/glutamate (1st succinate—rotenone). Complex II respiration was defined as respiration after adding the 2nd dose of succinate minus Complex I respiration. Average rates of oxygen consumption [(pmol/(s*ml)/μg protein]+ standard error of the mean (SEM) were graphed.

### Cold Enzyme Biopsy Prep to Generate Single Cells

Colon biopsies were minced in a Petri dish on ice in the presence of Native Bacillus Licheniformis psychrophilic proteases at 1 mg/ml (Creative Enzymes, Shirley, NY), transferred to an Eppendorf tube, intermittently vortexed for 30-60 seconds, placed on ice, and gently pipetted over 15 min. The suspension was centrifuged at 90 g and the supernatant filtered over a 40 mcM filter. Additional enzyme was added to residual tissue and the procedure repeated for an additional 15 minutes. Cells were counted with trypan blue and 85%-99% viability was noted.

### JC1 Mitochondrial Membrane Potential Measurement

JC1 staining was performed on the above single cell isolations with flow cytometry using the JC-1 (5,5″,6,6″-tetrachloro-1,1″,3,3″-tetraethylbenzimidazolylcarbocyanine iodide, Molecular Probes, Inc. Eugene, Oreg.) reagent according to the manufacturer's instructions. In brief, JC-1 dye was added at 1 mcM to washed cells, and incubated for 20 minutes at 37° C., 5% CO₂. Cells were washed and CD45 APC-Cy7 (BD Bioscience, Franklin Lakes, N.J.) and EpCAM APC (BioLegend, San Diego, Calif.) antibodies were added for an additional 30 minutes at room temperature. Cells were washed, acquired on a Canto flow cytometer, and data were analyzed using DeNovo software. The MMP was calculated as the ratio of PE-MFI/FITC-MFI in EpCAM+ and CD45+ cells. As a positive control for the specificity of the assay we used 50 mcM of CCCP (carbonyl cyanide 3-chlorophenylhydrazone) to depolarize the mitochondrial membrane potential measured using the JC-1 dye.

### Immunohistochemistry

Immunohistochemistry detection of MT-COL COX5A, and REG1A was performed using anti-Complex IV subunit I (Thermo Fisher Scientific cat. #459600), anti-Complex IV subunit Va (Thermo Fisher Scientific cat. #459120), and anti-REG1A (R&D Systems, INC. cat. #MAB4937). Staining was examined using an Olympus BX51 light microscope and digitally recorded at 20× and 40× magnification.

### Regression Analysis for Week 4 Remission

Multiple logistic regression was used to 1) determine the prognostic power of baseline clinical information, and 2) assess additional prognostic power resulting from including baseline gene expression in predicting remission 4 weeks after diagnosis in the moderate-severe group that received initial corticosteroid therapy. Pairwise association testing was performed to identify baseline variables appropriate for model building (nominal p-value<0.05). Clinical information considered for inclusion in the models were baseline clinical and endoscopic severity (Total Mayo EEF), Paris and Montreal classifications, presence of >32 eosinophils in the baseline rectal biopsy, gender, race, age at diagnosis, baseline BMI z-score, and serum albumin. The corticosteroid response genes PC1 and several other central genes pathways PC1 pre-identified by the previous differential expression analyses were considered, together with functional annotation enrichment analyses of the core 5296 UC genes and the 712 severity genes. The corticosteroid responsiveness gene signature passed a predefined expression filtering with the highest significance. For validation of the within subject biopsy consistency, parallel mRNAseq of paired biopsies obtained at the same time as the rectal sample used to derive the predictive gene panel in a subset of patients (n=6) were performed. Those comparison showed a strong correlation of 0.94 (P=0.005) for the corticosteroid responsiveness gene signature PC1 between pairs of biopsies.

Using forward selection, several logistic regression models were constructed. These models respectively include clinical and endoscopic severity, eosinophilic grade, and sex (model 1), and clinical and endoscopic severity, eosinophilic grade, sex, and the corticosteroid responsiveness gene signature PC1 (model 2). Model 3 tested how well eosinophil associated genes can replace the histologic eosinophil grade in model 2. At each step of model building, variables with p<0.1 were considered for inclusion; a likelihood ratio test was performed to compare the model with and without the new variable. Each new variable with likelihood ratio p<0.05 was maintained in the model. The reliability of the final model was tested by 10-fold cross validation. Model fit and improvement at each stage was assessed using AUC, Akaike Information Criterion (which penalizes for model complexity), and sensitivity and specificity.

### Summary of Statistical Tests Used

Shapiro-Wilk normality test was used on the continuous clinical parameters, and on specific gene expression, and PC1. If the data were not normally distributed, Mann-Whitney was used to compare two groups, and Kruskal-Wallis with Dunn's Multiple Comparison test was used for comparison of more than two groups. However, if the data were normally distributed unpaired t-test was used to compare two groups, and ANOVA with false discovery rate (FDR) was used for comparison of more than two groups. *All 2-sided P<0.05, **P<0.01, ***P<0.001. All statistical analyses were performed in SASv9.3 or GraphPad Prism v7.04.

### Results

(i) A Unique Treatment-Naive UC Inception Cohort

The PROTECT study systematically examined response of 428 newly diagnosed pediatric UC patients to consensus-defined disease severity-based treatment regimens guided by the Pediatric Ulcerative Colitis Activity Index (PUCAI). mRNA-Seq defined pre-treatment rectal gene expression for a representative discovery group of 206 UC PROTECT patients, a validation group of 50 UC PROTECT patients, and 20 age and sex matched non-IBD controls (see Table 1 above). The validation group had similar characteristics to the discovery group, but with a higher frequency of non-white participants. More severe endoscopic disease (Grade 3 Mayo endoscopic sub score, Chi squares p<0.001) and more extensive disease or pancolitis (Chi squares p<0.001) were noted in moderate-severe cases. Of the patients with mild disease, 53(98%) of 54 received initial therapy with mesalamine, and all moderate-severe patients received initial therapy with corticosteroids. Week 4 remission was defined as PUCAI<10 without additional therapy or colectomy and was achieved by 105 of 206 (51%) patients in the discovery cohort. 156 also had 16S rRNA sequencing to characterize their gut microbial communities.

(ii) The Core UC Gene Signature

A core rectal UC gene expression signature was identified in this study. The core rectal UC gene expression signature contains as many as 5296 genes differentially expressed [FDR<0.001 and fold change (FC)≥1.5] in comparison to controls (Ctl). Functional annotation enrichment analyses using ToppGene, ToppCluster, and CluGO mapped groups of related genes to biological processes. Chen et al., Nucleic acids research 37:W305-311, (2009); Kaimal et al., Nucleic acids research 38: W96-102 (2010); Bindea et al., Bioinformatics 25:1091-1093 (2009); and Haberman et al., The Journal of clinical investigation 124: 3617-3633 (2014).

Results showed highest enrichment for increased lymphocyte activation and associated cytokine signaling, and a robust decrease in mitochondrion, aerobic tricarboxylic acid (TCA) cycle, and metabolic functions. P values for the top specific biological processes were obtained as an output from ToppGene. Up-regulated gene signatures were enriched for integrin signaling (P<1.08E-12), JAK-STAT cascade, and TNF production (P<9.9E-93), pathways that are already associated with therapeutic advances in UC. Flamant et al., Drugs 77:1057-1068 (2017); and Abraham et al., Gastroenterology 152:374-388 (2017).

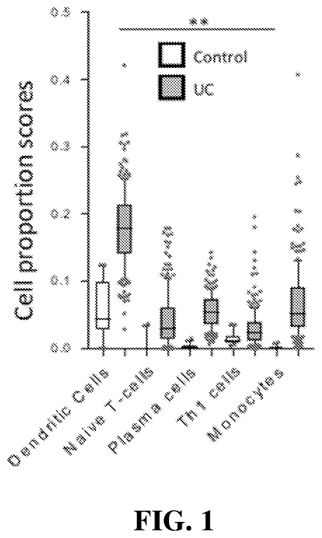

The down-regulated UC signature showed a robust decrease of mitochondrial-encoded and nuclear-encoded mitochondrial genes (P<2.76E-35). Applying a computational gene expression deconvolution approach to estimate the relative composition of immune cell subsets, epithelia, and other stromal cell types in each sample (see Methods above), showed a significant increase in the estimated proportion of several immune cells including T and B cells, dendritic cells (DC), and monocytes. FIG. 1. Using RISK cohort rectal biopsies mRNAseq data for treatment naïve pediatric UC patients and colonic biopsies microarray data of adults with active UC (GSE5907112), it was demonstrated that 87% of the differentially expressed genes in RISK UC, and 80% of the adult UC genes, were within the core PROTECT signature. Comparing the differentially expressed genes from isolated intestinal epithelial cells (IEC) from another pediatric UC inception cohort showed an overlap of 94% of the genes with the PROTECT genes, validating the majority of the core PROTECT UC signature in whole biopsies and in isolated epithelia.

Functional annotation enrichment analyses of the shared genes further confirmed many of the common enriched pathways. Comparing the shared down-regulated genes and pathways between PROTECT, RISK, adult UC cohort GSE5907112 (Vanhove et al., Inflammatory bowel diseases 21:2673-2682 (2015)), and the IEC UC cohort13 using ToppGene/ToppCluster confirmed the reduction of mitochondrial metabolic associated genes and pathways, genes associated with lipid metabolism, and genes associated with formation of adenoma and adenocarcinoma.

(iii) Robust Colonic Mitochondriopathy in UC.

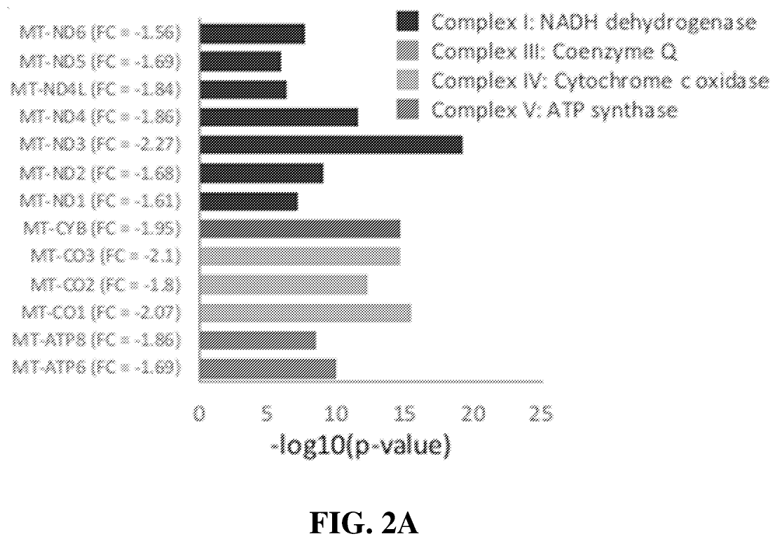

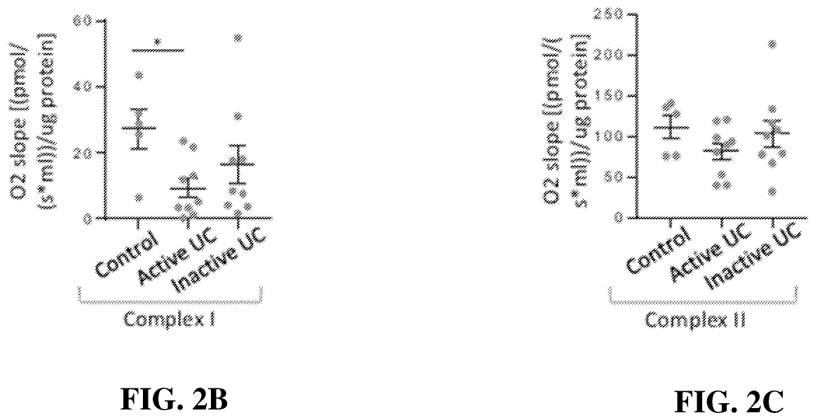

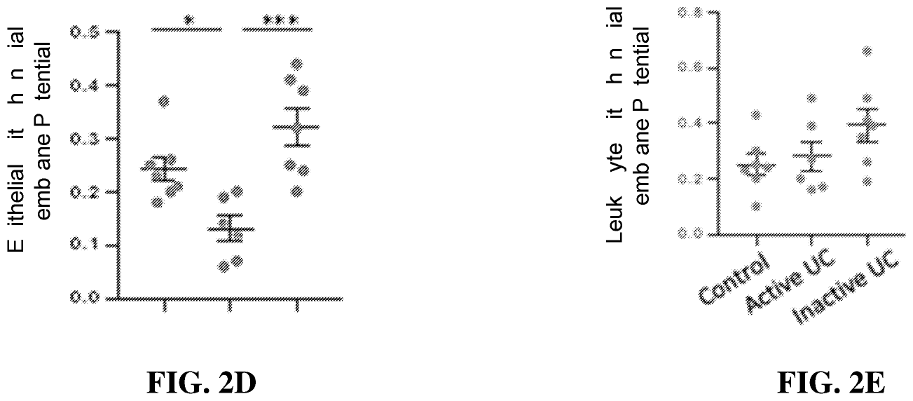

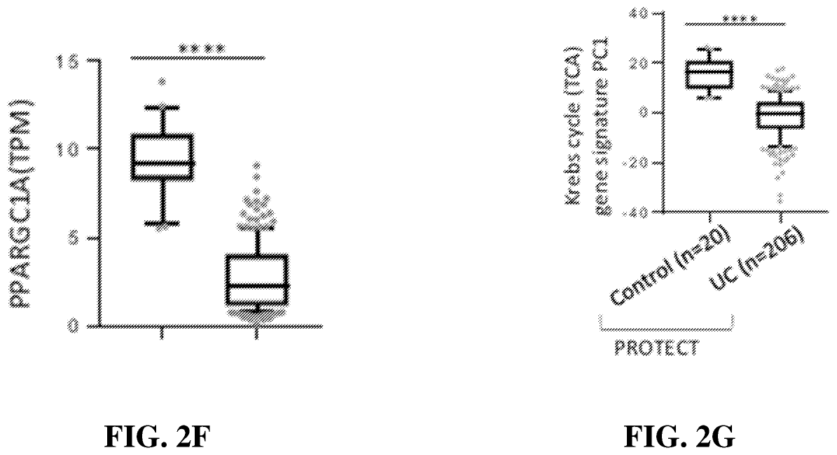

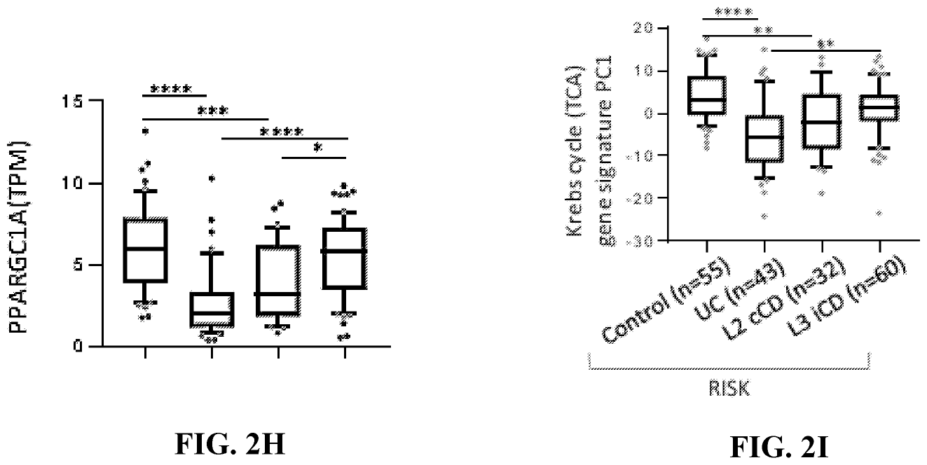

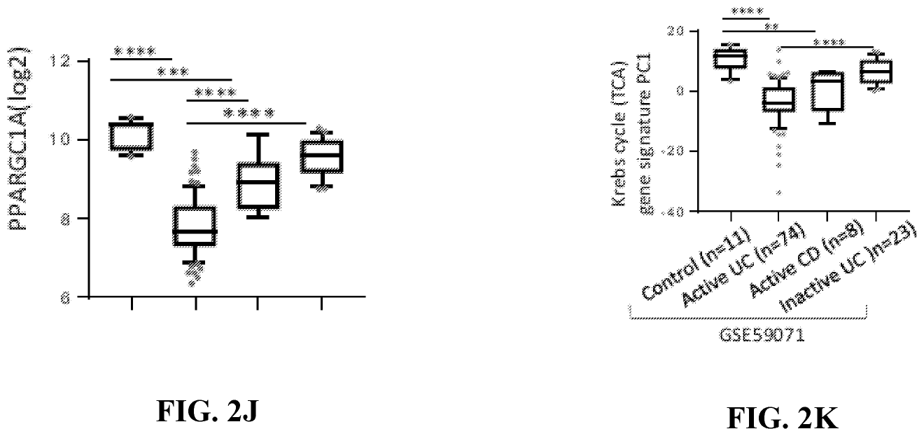

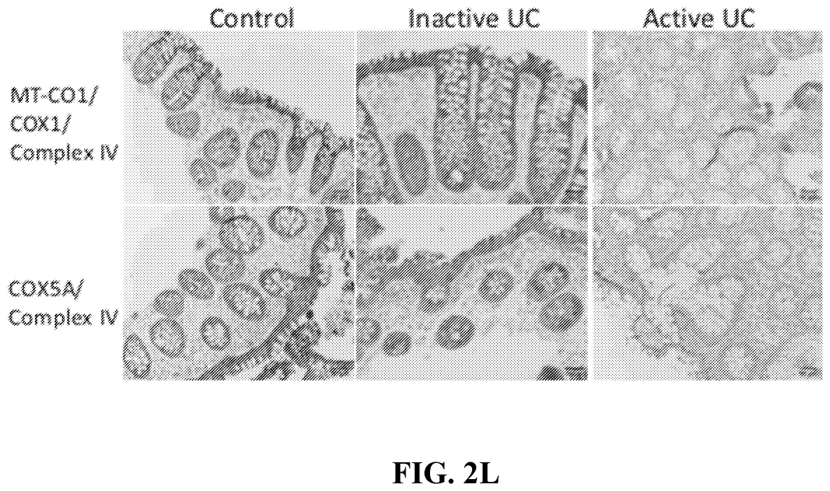

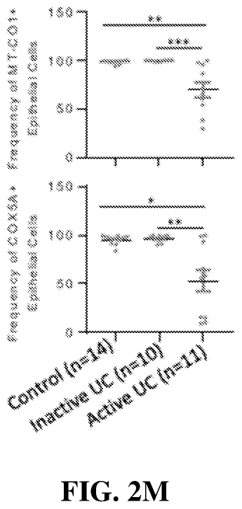

Notably, the mitochondrial genome encodes 13 genes regulating ATP production and all 13 were significantly reduced in UC. FIG. 2A. Real-time analysis of cellular respiration was subsequently evaluated in colonic biopsies from UC and control patients. Pesta et al., Methods in molecular biology 810: 25-58 (2012). Mitochondrial electron transport chain Complex I activity, the rate-limiting step in oxidative phosphorylation (Zielinski et al., Mitochondrion 31: 45-55 (2016); and Hroudova et al., Neural regeneration research 8: 363-375 (2013)) was reduced in active UC rectal biopsies compared to those from control patients. FIG. 2B. There was also a trend toward a decrease in Complex II activity. FIG. 2C. The mitochondrial membrane potential (MMP) that provides an integrated measure of the cellular capacity for ATP production was measured using JC-1 staining and FACS analysis of freshly isolated EpCAM+ colon epithelial cells (FIG. 2D) and CD45+ leukocytes (FIG. 2E). A specific reduction of MMP in epithelial cells was seen in active UC, with recovery in inactive UC. The mitochondrial membrane potential (MMP) in EpCAM+ epithelial cells and CD45+ leukocytes isolated from colon biopsies was measured using JC1 staining of rectal biopsy single cell preps and flow cytometry as shown (5,5″,6,6″-tetrachloro-1,1″,3,3″-tetraethylbenzimidazolylcarbocyanine iodide, Molecular Probes, Inc.).

As a positive control we stained cells with 1 mcM JC1 with and without the addition of 50 mcM of the depolarizing agent CCCP (carbonyl cyanide 3-chlorophenylhydrazone). In the JC1+CCCP cells there is a substantial reduction in the MMP, confirming the specificity of the JC1 alone result. The MMP was calculated as the ratio of PE-MFI/FITC-MFI in EpCAM+ and CD45+ cells. Representative FACS analyses of rectal biopsy single cell preps show the EpCAM+ epithelial and CD45+ leukocyte populations, with a marked increase in CD45+ cells in the active UC inflamed tissue. Mean fractions of control EpCAM+ epithelial cells and CD45+ leukocytes were 82% and 18%, in inactive UC were 71% and 29%, and in active UC 39% and 61%, respectively.

In addition, PPARGC1A (PGC-1α), the master regulator of mitochondrial biogenesis, was profoundly reduced in UC patients in comparison to controls in PROTECT, RISK, and adult UC (FIGS. 2F, 2H, and 2J), and the IEC UC cohort. Howell et al., 2017. Principal Coordinates Analysis (PCA) principal components 1 (PC1) to summarize the Krebs cycle (TCA) genes variations between patients showed reduction of genes regulating mitochondrial energy production in the UC groups (FIGS. 2G, 2I, and 2K). The RISK dataset revealed a spectrum of mitochondrial gene expression down-regulation in inflamed whole rectal biopsies, ranging from no significant suppression in mucosal biopsies obtained from inflamed rectum of ileo-colonic CD (L3 iCD) patients, to moderate suppression in samples from inflamed rectal biopsies of colon-only CD (L2 cCD) patients, and profound suppression in samples from pediatric UC samples with inflamed rectum (FIGS. 2H and 2I). The spectrum between UC and CD was validated in the adult IBD cohort (GSE5907112, FIGS. 2J and 2K). It was noted a recovery of this pathway in inactive adult UC. However, the larger PROTECT mRNAseq cohort permitted identification of an additional 3106 differentially expressed genes, which primarily demonstrated more robustly the suppression of mitochondrial pathways Immunohistochemistry confirmed reduced epithelial abundance of both mitochondrial encoded MT-CO1 and nuclear encoded COX5A genes, which comprise complex IV in active UC (FIGS. 2L and 2M).

(iv) Disease Severity Gene Signatures.

More severe disease is linked in the data reported herein and others to higher rates of therapy escalation and colectomy, whereas mild disease is associated with remission by 12 weeks. Hyams et al., 2017; and Turner et al., Gastroenterology 138:2282-2291, (2010). Unsupervised hierarchical clustering analysis using the core 5296 genes grouped 204 of 206 UC cases in the dendogram cluster A while all 20 non-IBD controls were in cluster B. Most mild cases grouped in A(i), while severe cases tended to be enriched in cluster A(ii) (P<0.001). The core UC 5296 gene principle component 1 (PC1) values separated Ctl from UC across both clinical and endoscopic severity, while PC2 contributed to separation within UC severity. 106 genes were significantly differentially expressed between severe vs. moderate and between moderate vs. mild UC clinical disease defined by PUCAI, showing stepwise alteration across cases. 916 genes were identified as differentially expressed between UC with severe vs. mild clinical disease and 1038 genes were identified as differentially expressed between severe vs. mild endoscopic sub score (FDR<0.001 and FC≥1.5). An overlap of 712 genes (292 down- and 420 up-regulated genes) results relative to the core UC signature, referred to hereafter as the UC severity signature.

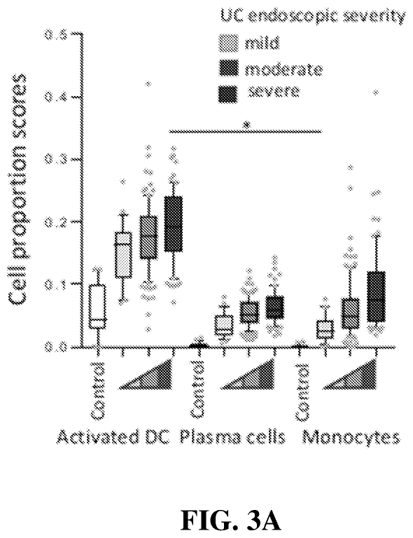

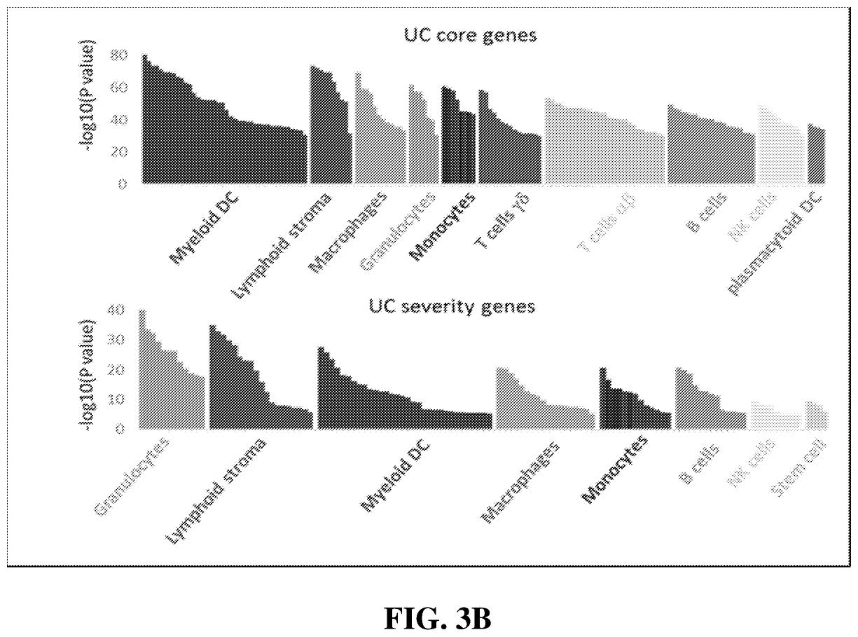

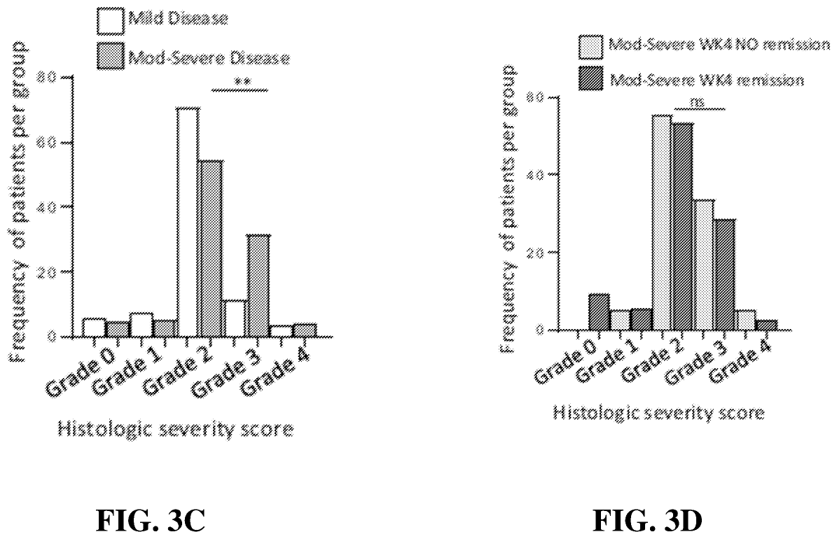

Functional annotation enrichment analyses of the UC severity signature emphasized genes that are down- (P<4.54E-46) and up-regulated (P<7.62E-51) in colorectal adenoma. Immunohistochemistry confirmed increased epithelial abundance of REG1A gene, known to be upregulated in both UC and in colitis-associated colorectal cancer (CAC) 18 in active UC. In addition, up-regulated severity genes were also enriched for innate immunity (P<7.07E-19), neutrophil degranulation (P<1.51E-16), and CXCR1 interactions (P<9.08E-8). Relative composition of immune cell subsets using a computational gene expression deconvolution approach showed an increase in activated DC, plasma cells, and monocytes in patients with severe vs. mild disease. FIG. 3A. An alternative analytic approach using the Immunological Genome Project data series as a reference through ToppGene also identified an increased proportion of myeloid cells with increased severity. FIG. 3B.

(v) Rectal Genes Correlated with Histologic Features.

Rectal biopsy histology was evaluated centrally. Surface villiform architectural abnormality was linked to escalation therapy or colectomy. Hyams et a., 2017; and Boyle et al., 2017. Hematoxylin and eosin (H&E, 100×) staining of control and UC case with acute cryptitis, showed crypts that do not rest on the muscularis mucosa, and marked surface villiform change. 187 genes (69 up- and 118 down-regulated) were identified as differentially expressed (FDR<0.001 and FC≥1.5) between UC patients with and without surface villiform changes. Most of these genes overlapped with the 712 UC severity genes, suggesting a molecular link between this histologic feature and UC severity. In contrast, higher eosinophil infiltrate (>32 rectal eosinophils/hpf,) was associated with a favorable week 12 outcome. Hyams et a., 2017; and Boyle et al., 2017. Three genes differed significantly (FDR<0.001 and FC≥1.5) between UC patients with and without higher infiltrating eosinophils. This included Arachidonate 15-Lipoxygenase (ALOX15) involved in production of lipid mediators, which resolve inflammation. A Histologic Severity Score for chronic and active acute neutrophil inflammation was defined as follows: grade 0=no inflammation, grade 1=chronic inflammation only, grade 2=mild acute neutrophil inflammation—no crypt abscesses, grade 3=moderate to marked acute neutrophil inflammation with crypt abscesses, and grade 4=Mucosal ulcers and erosions. Boyle et al., 2017.

While a higher frequency of patients with moderate-severe disease was noted to show marked acute inflammation with crypt abscesses (grade 3) histology than the frequency noted within patients with mild disease (FIG. 3C), no such difference was noted within moderate-severe patients that did or did not achieve week 4 (WK4) remission (FIG. 3D).

(vi) Corticosteroid Responsiveness Gene Signature and Microbial Shifts.

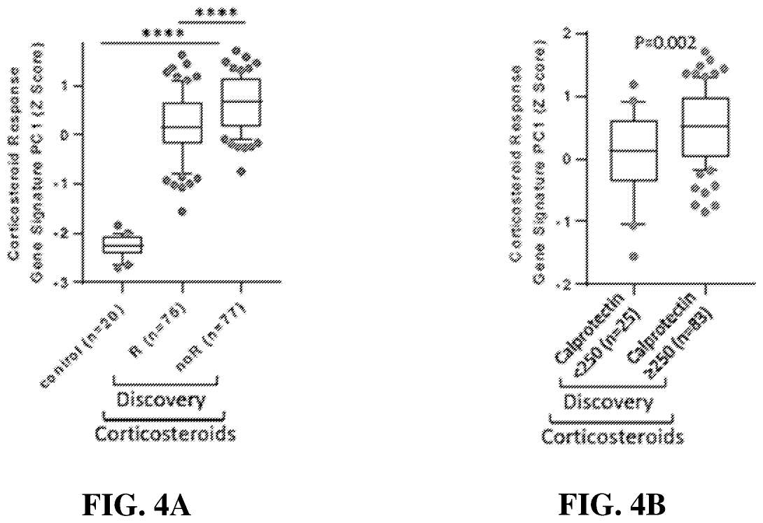

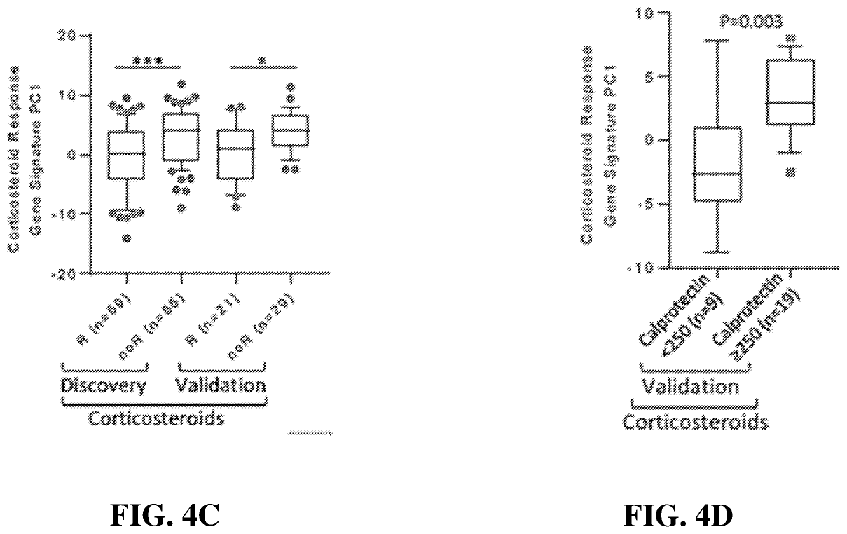

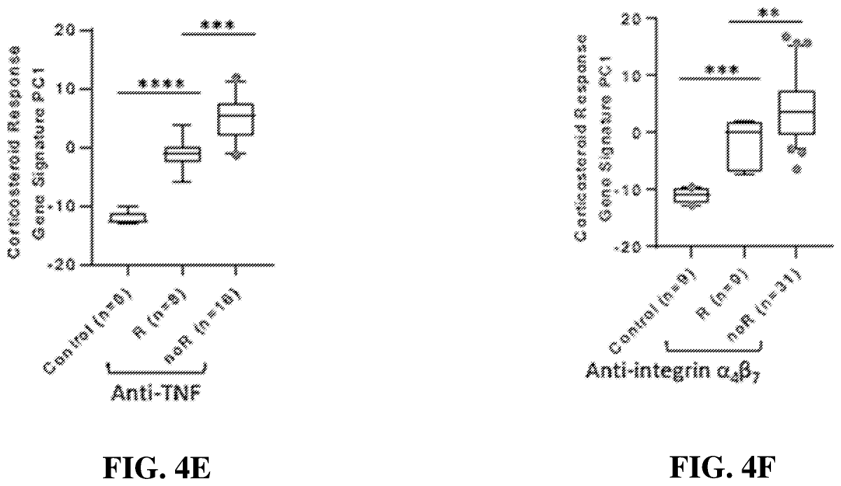

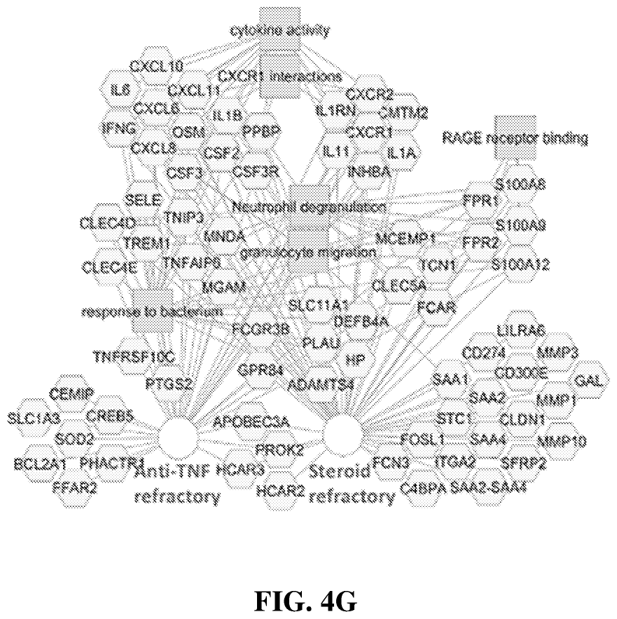

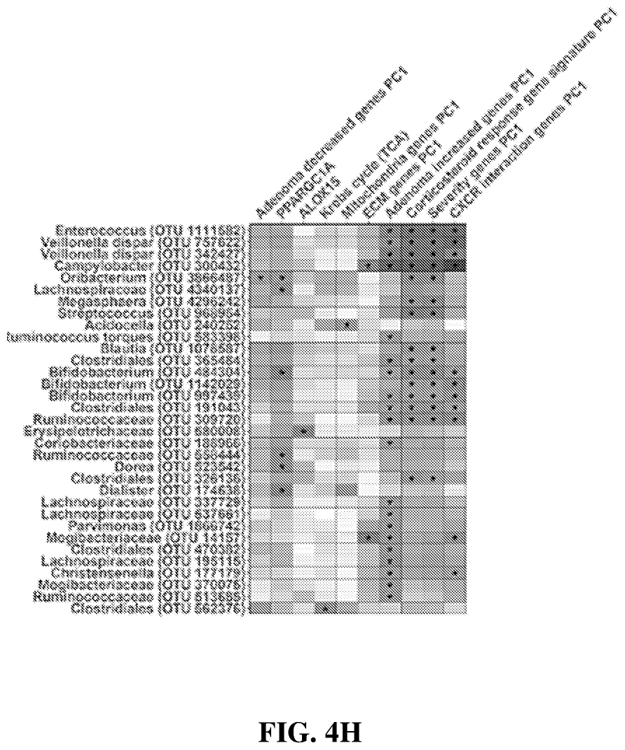

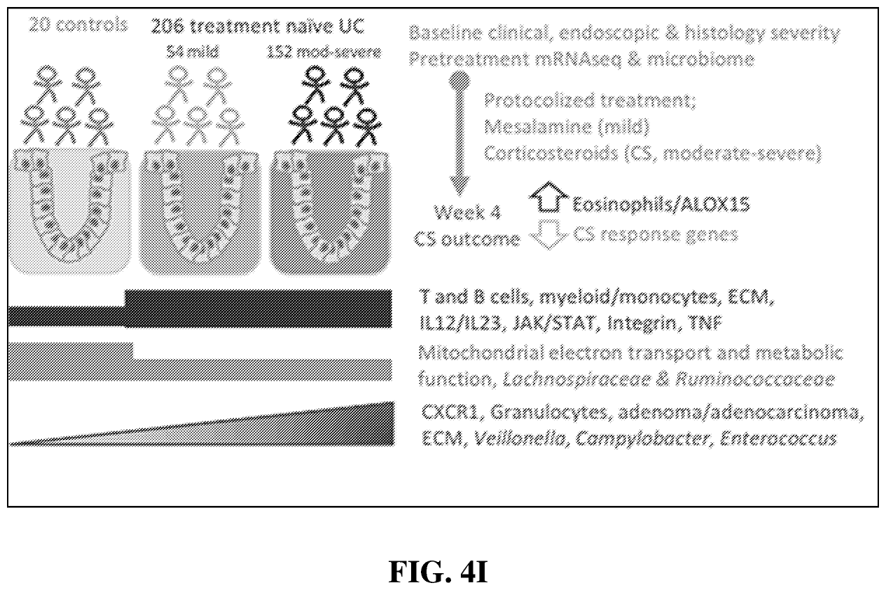

In the full cohort, the strongest predictor of corticosteroid-free remission by week 12 was clinical remission at week 4 (WK4), irrespective of initial corticosteroid status. Hyams et al., 2017. When considering WK4 remission, clinical factors associated with this outcome included disease severity and rectal biopsy eosinophil count. Based on these results, the analysis was focused on the WK4 outcome of moderate-severe patients that received corticosteroids. A corticosteroid responsiveness gene signature composed of 115 differentially expressed genes (FDR<0.05 and FC≥1.5) in baseline rectal biopsies between moderate-severe UC patients who did or did not achieve WK4 remission was defined (FIGS. 4A-I, and Table 1 above). The corticosteroid responsiveness gene signature (115 genes) originated from differential expression between moderate-severe patients that achieved Week 4 (Wk4) remission and those that did not of the 712 severity genes. Computational deconvolution analysis of cell subset proportions in controls and moderate-severe UC patients that did or did not achieve week 4 remission within the cells were examined Only the monocyte cell proportion exhibited a significant difference between UC patients stratified by week 4 remission in Kruskal-Wallis with Dunn's Multiple Comparison test.

PCA PC1 values summarized variation in the corticosteroid responsiveness gene signature which was differentially expressed based on Week 4 clinical remission (R vs NoR, FIG. 4A), and week 4 mucosal healing defined as fecal calprotectin <250 mcg/gm (FIG. 4B) in the Illumina discovery cohort. Healthy controls showing lower scores, implying that patients destined to respond to CS have a more healthy profile with respect to this gene signature at baseline. The corticosteroid responsiveness gene signature PC1 was replicated using the Lexogen platform (Tuerk et al., PLoS Comput Biol 13:e1005515 (2017)) in the subset of 134 UC patients with Illumina data, as well an independent sub-cohort of 50 UC patients that were not included in the original analysis (FIGS. 4C and 4D). As there are no other mucosal transcriptomic studies that examined response to standardized initial corticosteroid induction therapy, we tested previous transcriptomic studies that examined anti-TNF (GSE1687920) or anti-integrin α4β7 (GSE7366123) response. Arijs et al., PloS one 4:e7984, (2009); West et al., Nat Med 23:579-589 (2017); Gaujoux et al., Gut, doi:10.1136/gutjnl-2017-315494 (2018); and Arijs et al., Gut 67:43-52 (2018). A similar difference with anti-TNF or anti-integrin α4β7 response in adult UC was noted as defined by mucosal healing at colonoscopy (FIGS. 4E and 4F).

Interestingly, Oncostatim M (OSM; West et al., 2017) and TREM1 (Bindea et al., 2009) previously associated with anti-TNF response, were within our corticosteroid responsiveness gene signature (FIG. 4G), and this signature PC1 showed a high correlation with OSM and TREM1 (0.79 and 0.89, P<0.0001). A substantial overlap between the genes from the PROTECT corticosteroid responsiveness gene signature and previously described anti-TNF response genes was noted. FIG. 4G.

Functional annotation enrichment analyses of the corticosteroid responsiveness gene signature were performed and the full output from ToppGene (Table 2) with more detailed ToppCluster output is shown in FIG. 4G. Those analyses indicated that this signature is highly associated with cytokines including CXCR (P<7.12E-12), innate myeloid immune signatures (P<1.62E-15), and response to bacteria (P<2.16E-13). Aberrant immune responses to shifts in commensal microbes likely play a role in UC pathogenesis and treatment responses. 152 of the 206 UC patients in our cohort also had fecal 16S rRNA microbial profiles. By applying hierarchical all-against-all association testing MAHAL genes and pathways associated with specific microbial Operational Taxonomic Units (OTUs) were identified, including associations between disease severity associated taxa such as Campylobacter, Veillonella, and Enterococcus with genes and pathways linked to a more severe disease form, and refractory disease in connection with initial corticosteroid induction therapy. In contrast, decreased taxa from the Clostridiales order that are considered beneficial were identified, which show a negative correlation with gene signatures associated with disease severity and unfavorable treatment responses. FIG. 4H.

(vii) Gene Signatures Improve Prediction of Week 4 Remission

It was further explored whether gene expression data would improve a multivariable regression WK4 prediction model based on clinical factors alone (Table 3). A model that included (Table 3, model 1) sex, disease severity (total Mayo clinical and endoscopic severity score), and histologic characterization of rectal eosinophils agreed with the model for the full cohort, adding sex with borderline significance. The corticosteroid responsiveness gene signature PC1 was negatively associated with Week 4 outcome (model 2, OR 0.36, 95% CI 0.18-0.71; p=0.003). When this gene signature was included, the AUC improved to 0.774 (Likelihood ration p-value <0.002), indicating superiority to the model which included clinical factors alone. In model 3, the eosinophil count was replaced with the eosinophil-associated gene ALOX15 without harming the model accuracy with some improvement of the discriminant power (AUC of 0.777, 0.692-0.848), sensitivity of 62.7%, (95% CI 52.8-72.5%), specificity of 76.6% (95% CI 0.68.8%-84.4%), positive predictive value of 72.3%, and negative predictive value of 67.8% (AUC cutoff at ≥0.5). Bootstrapping and multiple imputation were used for internal validation and were generally supportive of the final selected moderate/severe model. The Histologic Severity Score (HSS) showed moderate correlation with the corticosteroid responsiveness gene signature PC1 (Spearman r=0.31, p<0.001), but not with WK4 outcome. Moreover, the gene signature was still significant in the model even after adjusting for the HSS. Similarly, while the monocyte deconvolution score showed high correlation with the corticosteroid responsiveness gene signature PC1 (Pearson r=0.72, P<0.001) and was different between WK4 responders and non-responders, it was not significant when added to the model in place of the gene signature, while the gene signature remained significant in the model after adjusting for the monocyte score.

TABLE 3
Multivariable Models of Baseline Characteristics and Gene Expression Associated with Week 4 Remission in 147 Patients
with Moderate-severe Disease that Received Corticosteroids.

| Model # | Model Variables | OR (95% CI) | Variable P | Model AIC | Model AUC | Model ChiSq | Model P |
|---|---|---|---|---|---|---|---|
| 1 | Total Mayo Score (range 0-12) | 0.68 (0.54-0.85) | 0.0007 | 186.03 | 73.7 | 25.75 | <0.0001 |
| | Rectal Eosinophil Level | 2.27 (1.11-4.63) | 0.0245 | | (65.4-82.0) | | |
| | (count > 32/hpf) | | | | | | |
| | Sex (M vs F) | 0.47 (0.22-0.99) | 0.039 | | | | |
| 2 | Total Mayo Score (range 0-12) | 0.77 (0.61-0.98) | 0.032 | 178.51 | 77.4 | 35.27 | <0.0001 |
| | Rectal Eosinophil Level | 1.81 (0.85-3.84) | 0.122 | | (69.7-85.1) | | |
| | (count > 32/hpf) | | | | | | |
| | Sex (M vs F) | 0.47 (0.22-0.99) | 0.048 | | | | |
| | Corticosteroid Responsiveness | 0.36 (0.18-0.71) | 0.003 | | | | |
| | gene signature (PC1 z-score values) | | | | | | |
| 3 | Total Mayo Score (range 0-12) | 0.79 (0.63-1.00) | 0.055 | 172.98 | 77.7 | 40.80 | <0.0001 |
| | ALOX15 Gene Exp. (TPM) | 2.59 (1.21-5.52) | 0.014 | | (70.0-85.4) | | |
| | Sex (M vs F) | 0.45 (0.21-0.96) | 0.038 | | | | |
| | Corticosteroid Responsiveness gene | 0.40 (0.2-0.79) | 0.009 | | | | |
| | signature (PC1 z-score values) | | | | | | |

OR: odds ratio;
AIC: Akaike’s information criterion;
AUC: area under the ROC curve;
LR: likelihood ratio;
ROC: Receiver Operator Characteristic.
LR = 9.519 and LR P-value = 0.002 when comparing model 2 to model 1.

### CONCLUSIONS

PROTECT is the largest prospective inception cohort study to examine factors associated with early responses to standardized first-line therapy in pediatric UC. This study provided evidence for core host gene expression profiles driving lymphocyte activation and cytokine signaling which are targeted by current therapies. The data also suggested a robust reduction in epithelial mitochondrial genes and associated energy production pathways in UC, which were not directly addressed by current approaches. This reduction of mitochondrial genes was validated in treatment naïve pediatric UC, adults with active UC with longstanding disease, and more specifically in viable isolated epithelia of treatment naïve pediatric UC. Genes and pathways that are linked to UC severity were captured and those regulating epithelial transformation and innate CXCR-mediated leukocyte recruitment were prioritized. A gene signature linked to corticosteroid response was identified, which was validated in an independent subset of UC patients, and showed substantial overlap with genes previously associated with anti-TNF response. A multivariable analysis combining the corticosteroid responsiveness gene signature PC1 and ALOX15 gene expression with clinical variables better predicted corticosteroid responsiveness than clinical factors alone. These findings are summarized in FIG. 4I.

Decreased mitochondrial activity was previously described in UC, but understanding of the molecular mechanism was lacking. Sifroni et al., Mol Cell Biochem 342: 111-115, (2010); Santhanam et al., Inflammatory bowel diseases 18:2158-2168 (2012); Mottawea et al., Nature communications 7:13419 (2016); Cardinale et al., PloS one 9:e96153 (2014); Palsson-McDermott et al., Cell Metab 21:65-80 (2015); and Hoshi et al., Science 356: 513-519 (2017). Dysfunctional mitochondria exacerbate barrier dysfunction and inflammation, while pro-29 and anti-30 inflammatory stimuli affect mitochondrial metabolic functions. PPARGC1A (PGC1α), the master regulator of mitochondrial biogenesis, ameliorated experimental colitis, whereby intestinal epithelial depletion of PGC1α suppressed mitochondrial function and the intestinal barrier. Cunningham et al., The Journal of biological chemistry 291:10184-10200 (2016). Mitochondrial loss also preceded the development of colonic dysplasia in UC, and high mitochondrial activity reflecting electron transport in the ileum was also associated with protection against CD progression in RISK. Ussakli et al., Journal of the National Cancer Institute 105:1239-1248 (2013); and Kugathasan et al., Lancet, doi:10.1016/S0140-6736(17)30317-3 (2017).

It was reported here a substantial suppression of all 13 electron transport mitochondrial-encoded genes (Complex I, III, IV, and V), PPARGC1A (PGC1α), and epithelial mitochondrial membrane potential, which further supported the robustness of the colonic mitochondriopathy in UC. Moreover, it was demonstrated that specificity of mitochondrial gene expression down-regulation in colon-only forms of IBD rather than in CD patients with both ileal and colonic inflammation. Peterson et al., Parasitology international 60:296-300 (2011); and Schieffer et al., American journal of physiology. Gastrointestinal and liver physiology 313:G277-G284 (2017). Interestingly, previous studies in infectious colitis or diverticulitis demonstrated an induction of immune and wound healing genes, with considerable overlap with the immune and wound healing genes identified in pediatric UC for the current report. However, these studies did not demonstrate a similar reduction in mitochondrial genes, suggesting specificity of this response in UC.

Functionally, a decrease in the activity of Complex I of the electron transport chain in the inflamed rectums of patients with UC was observed, as well as a reduction of mitochondrial depolarization more specifically in epithelia. Although a defect in respiration has been observed in the colons of UC patients previously, mitochondrial function from intestinal biopsies has not been reported before been evaluated via high-resolution respirometry. With real-time analysis of intact human tissue, this technique offers precise evaluation of mitochondrial membrane integrity and oxidative capacity. In conjunction with the expression data, these results suggest a downregulation and dysfunction of mitochondrial respiration, characterized by a defect at Complex I, the rate-limiting step in oxidative phosphorylation. Supplementing the mitochondrial electron transport axis via medical, environmental, or nutritional approaches can be potential targets for future therapies.

Inflammation has a substantial cumulative role in colitis-associated colorectal cancer (CA CRC) development and is closely linked to the extent, duration and severity. Ekbom et al., The New England journal of medicine 323:1228-1233, (1990); Eaden et al., Gut 48:526-535 (2001); and Rutter et al., Gastroenterology 130:1030-1038 (2006). Studies in the noncancerous IBD mucosa indicated that colorectal cancer development in IBD begins many years before the development of neoplasia as part of the occult evolution within the inflamed bowel. Choi et al., Nature reviews. Gastroenterology & hepatology 14:218-229 (2017). Here, a profound dysregulation of gene sets was detected as associated with disease severity previously implicated in adenocarcinoma. The results therefore showed that not only at the genomic and epigenetic level, but also at the transcriptomic level, already at diagnosis, genes and pathways that are associated with UC severity show associations with epithelial transformation. Choi et al., Nature reviews. Gastroenterology & hepatology 14:218-229, (2017); and Leedham et al., Gastroenterology 136:542-550 e546 (2009).

Microbial organisms and products affect host immune education, development and response, and aberrant immune responses to commensal microbes likely contribute to gut inflammation which is the hallmark of UC. Sartor et al., Gastroenterology 152:327-339 (2017). This study showed positive associations between genes and pathways associated with UC severity and response to treatment and disease-linked microbial taxa. Negative associations involved more beneficial commensal taxa with pathways and genes that were linked to resolution of inflammation or up-regulated in non-IBD controls. Those included oral pathobionts Veillonela dispar, and Campylobacter, and depletion of several commensal organisms such as Lachnospiraceae, Bifidobacterium, and Ruminococcaceae suggesting a substantial depletion of SCFA-producing bacteria that may affect epithelial barrier function. Kelly et al., Cell host & microbe 17:662-671 (2015).

In this study and in previous studies in children and adults, higher baseline disease severity identified patients less likely to achieve remission with corticosteroids. Romberg-Camps et al., The American journal of gastroenterology 104:371-383 (2009); and Moore et al., Inflammatory bowel diseases 17:15-21 (2011). The instant results supplemented and improved those models by adding baseline gene expression data. A gene signature linked to corticosteroid response was identified and validated in an independent subset of UC patients. The corticosteroid responsiveness gene signature is enriched for cytokines (CXCR1/2) and chemokines CXCL/6/8/10/11/17, which promote activation of the innate immune system and recruitment of neutrophils, and to response to external stimuli and bacteria. Notably, the corticosteroid responsiveness gene signature showed a substantial overlap with genes previously associated with anti-TNF response, and exhibited a similar difference between responders and non-responders to anti-TNF or anti-integrin α4β7 therapies. These similarities support an emerging concept in the field that the mucosal inflammatory state as measured by gene expression may better define the likelihood of response to current treatment approaches then conventional clinical measures of severity. By comparison, higher ALOX15 expression was linked to a higher likelihood for remission. Increasing evidence suggests a role for ALOX15 expressed in tissue eosinophils and macrophages in the resolution of inflammation, by interfering with neutrophil recruitment in models of arthritis, postoperative ileus, and peritonitis. Ackermann et al., Biochim Biophys Acta 1862:371-381 (2017); Chan et al., J Immunol 184:6418-6426 (2010); Stein et al., Journal of leukocyte biology 99:231-239 (2016); and Yamada et al., FASEB J 25:561-568 (2011).

In summary, the UC transcriptomics cohort reported herein is the largest and most comprehensive to date and the only data set to utilize pre-treatment samples, and to link these to 16S microbial community data and response to standardized first-line corticosteroid therapy. A robust colonic mitochondriopathy in overall UC pathogenesis was implicated. Already at diagnosis genes associated with UC severity are enriched for those known to drive epithelial transformation. A validated corticosteroid responsiveness gene signature and higher anti-inflammatory ALOX15 expression are associated with higher odds of achieving early clinical remission, with remarkable over-lap with genes implicated in response to biologics. A shift to personalized approaches targeting specific mechanisms in individual patients would be key to reducing the increasing disease burden of UC worldwide.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one,

A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for assessing responsiveness to a ulcerative colitis (UC) therapy in a subject having UC, the method comprising:
(i) measuring expression levels of a group of genes in a biological sample of a subject having UC, wherein the group of genes consists of two or more genes selected from the genes listed in Table 1;
(ii) determining a steroid responsiveness gene signature based on the expression levels of the two or more genes in step (i); and
(iii) assessing the subject's responsiveness to a UC therapy based on at least the steroid responsiveness gene signature.

2. The method of claim 1, wherein the subject is a human pediatric patient having ulcerative colitis.

3. The method of claim 1, wherein the subject is free of steroid treatment.

4. The method of claim 1, wherein the group of genes comprises at least two genes involved in two different biological pathways, and wherein the two different biological pathways are selected from the group consisting of cytokine activity, CXCR1 interaction, RAGE receptor binding, neutrophil degranulation, granulocyte migration, and response to bacterium.

5. The method of claim 4, wherein the group of genes comprises at least one gene involved in cytokine activity, one gene involved in CXCR1 interaction, one gene involved in RAGE receptor binding, one gene involved in neutrophil degranulation, one gene involved in granulocyte migration, and one gene involved in response to bacterium.

6. The method of claim 1, wherein the group of genes comprise DEFB4A, CSF2, CXCR1, S100A9, FCGR3B, OSM, and TREM1.

7. The method of claim 1, wherein the group of genes consists of all genes listed in Table 1.

8. The method of claim 1, wherein the biological sample is a rectal biopsy sample of the subject.

9. The method of claim 1, wherein the expression levels of the group of genes are measured by RT-PCR and microarray analysis.

10. The method of claim 1, wherein the steroid responsiveness gene signature is determined by a computational analysis.

11. The method of claim 10, wherein the steroid responsiveness gene signature is represented by a score calculated by the computational analysis based on the expression levels of the group of genes, and wherein deviation of the score from a predetermined value indicates whether the subject would respond to or not respond to the UC therapy.

12. The method of claim 1, wherein in step (iii), assessment of the subject's responsiveness to the UC therapy is further based on one or more clinical factors.

13. The method of claim 12, wherein the one or more clinical factors comprise gender, level of rectal eosinophils, and disease severity.

14. The method of claim 13, wherein the level of rectal eosinophils is represented by the expression level of ALOX15 in a rectal biopsy sample of the subject.

15. The method of claim 1, wherein the UC therapy responsiveness comprises Week 4 clinical remission.

16. The method of claim 1, further comprising, prior to step (iii), analyzing microbial populations in the biological sample.

17. The method of claim 16, wherein in step (iii), assessment of the subject's responsiveness to the UC therapy is further based on abundance of disease-associated and beneficial microbial populations in the biological sample.

18. The method of claim 1, wherein the UC therapy comprises a steroid, an anti-TNFα agent, an anti-α4β7 integrin agent, or a combination thereof.

19. The method of claim 18, wherein the UC therapy comprises a steroid.

20. The method of claim 19, wherein the steroid is a corticosteroid.

21. The method of claim 1, further comprising subjecting the subject to a suitable treatment of ulcerative colitis based on the assessment of the subject's responsiveness to the UC therapy determined in step (iii).

22. The method of claim 1, wherein the subject is determined to be responsive to the UC therapy and the method further comprises administering to the subject a steroid, an anti-TNFα agent, an anti-α₄β₇ integrin agent, or a combination thereof, for treating ulcerative colitis.

23. The method of claim 22, wherein the subject is administered with a steroid.

24. The method of claim 23, wherein the steroid is a corticosteroid.

25. The method of claim 1, wherein the subject is determined to be non-responsive to the UC therapy and the method further comprises administering to the subject a non-steroid therapeutic agent for treating ulcerative colitis.

26. The method of claim 25, wherein the non-steroid therapeutic agent is neither an anti-TNFα agent nor an anti-α₄β₇ integrin agent.

27. A method for identifying a subject having or at risk for ulcerative colitis (UC), the method comprising:
(i) measuring expression levels of (a) one or more genes involved in mitochondrial function, (b) one or more genes involved in the Kreb cycle, or (c) a combination of (a) and (b), in a biological sample of a subject;
(ii) determining a UC disease occurrence and/or severity gene signature based on the expression levels of the genes in step (i); and
(iii) assessing UC occurrence or severity of the subject based on the gene signature determined in step (ii).

28. The method of claim 27, wherein the one or more genes involved in mitochondrial function comprises PPARGC1A (PGC-1α), MT-CO1, COX5A, a Complex I gene, a Complex III gene, a Complex IV gene, a Complex V gene, or a combination thereof.

29. The method of claim 28, wherein step (i) involves measuring the expression level of PPARGC1A (PGC-1α) in the biological sample.

30. The method of claim 27, wherein step (i) involves measuring the levels of MT-CO1⁺ and/or COX5A⁺ cells in the biological sample.

31. The method of claim 27, wherein step (i) involves measuring the level of the Complex I gene, the Complex III gene, the Complex IV gene, the Complex V gene, or a combination thereof.

32. The method of claim 28, wherein:
(a) the Complex I gene is MT-ND1, MT-ND2, MT-ND3, MT-ND4, MT-ND4L, MT-ND5, and/or MT-ND6,
(b) the Complex III gene is MT-CYB;
(c) the Complex IV gene is MT-CO1, MT-CO2, and/or MT-CO3; and/or
(d) the Complex V gene is MT-ATP6 and/or MT-ATPS.

33. The method of claim 27, wherein the biological sample is a rectal biopsy sample of the subject.

34. The method of claim 27, wherein the expression levels of the genes are measured by RT-PCR and microarray analysis.

35. The method of claim 27, wherein the UC disease occurrence and/or severity gene signature is determined by a computational analysis.

36. The method of claim 27, wherein the subject is identified as having or at risk for UC and the method further comprises subjecting the subject to a treatment of UC.

37. The method of claim 27, wherein the subject is a UC patient and is identified as having an active disease, and wherein the method further comprises subjecting the subject to a treatment of UC.

38. The method of claim 37, wherein the subject has undergone a prior treatment of UC and the method comprises administering to the subject at least one therapeutic agent that is different from the therapeutic agent(s) involved in the prior treatment.
